(19) United States
(12) Patent Application Publication
(10) Pub. No.: US 2023/0016983 A1
(43) Pub. Date: Jan. 19, 2023

(54) ANTISENSE OLIGONUCLEOTIDES AND THIER USE FOR THE TREATMENT OF CANCER

(71) Applicants: lNSERM (Institut National de la Santé et de la Recherche Médicale), Paris (FR); Centre National de la Recherche Scientifique (CNRS), Paris (FR); Université d'Aix Marseille, Marseille Cedex 07 (FR); Institut Jean Paoli & Irene Camettes, Marseille (FR)

(72) Inventors: Palma ROCCHI, Marseille Cedex 09 (FR); Thi-Khanh LE, Marseille Cedex 09 (FR); François BERTUCCI, Marseille Cedex 09 (FR); David TAIEB, Marseille Cedex 09 (FR); Clément PARIS, Marseille Cedex 09 (FR); Pascal FINETTI, Marseille Cedex 09 (FR)

(21) Appl. No.: 17/777,792

(22) PCT Filed: Nov. 18, 2020
(86) PCT No.: PCT/EP2020/082548

§ 371 Date: May 18, 2022

(30) Foreign Application Priority Data
Nov. 19, 2019 (EP) 19306485.4

(51) Int. Cl. C12N 15/113

(52) U.S. Cl. CPC C12N 15/1137; C12Y 306/04013; C12N 2310/11

(57) ABSTRACT

The present invention concerns the treatment of prostate cancer and particularly castration resistant prostate cancer (CRPC). The Heat Shock Protein Hsp27, a chaperone protein, has been long demonstrated as a driver of Castration Resistance Prostate Cancer (CRPC). In the light of identification of the molecular mechanisms, the inventor determined that the Probable ATP-dependent RNA helicase DDX5 is an interactor of Hsp27 and DDX5's expression is modulated by Hsp27. They confirmed that DDX5 overexpression is correlated to the aggressiveness of the tumor, to the CRPC emergency and to the biochemical recurrence risk. They also developed DDX5-targeting antisense oligonucleotides for research purpose and clinical application. Thus, the invention relates to an inhibitor of DDX5 wherein said inhibitor reduces the expression and/or activity of DDX5 in a subject in need thereof and targets the gene or the mRNA of DDX5.

### FIELD OF THE INVENTION

The present invention relates to antisense oligonucleotides efficiency in reducing the expression of DDX5 in cancer of a subject.

### BACKGROUND OF THE INVENTION

Cancer is the second leading cause of death globally. For instance, Prostate cancer (PC) is one of the most frequent cancers in developed countries. Patients with PC at the early state are consulted to apply surgery or radiation while androgen deprivation therapy (ADT) is used broadly in patients with advanced PC. Most of the PC victims originally raise positive responses to ADT; however, they finally turn into insensitive and relapse as castration-resistant PC (CRPC), a highly aggressive terminal stage of PC (Katsogiannou et al. 2015).

The inventors have been focusing on exploring mechanisms of CRPC in order to restore tumor sensitivity to difference therapies. They found out that Hsp27 was highly overexpressed after androgen ablation, resulting in protecting tumors from cellular stresses (Rocchi et al. 2004, 2005). Moreover, Hsp27 knock down by using siRNA and the oligonucleotide antisense OGX427 demonstrated to enhance the tumor sensitivity to castration and chemotherapy (Rocchi et al. 2005) (Rocchi et al. 2006). The antisense OGX427 (Apatorsen, Patent PCT no 10/605, 498, 2005) has finished the phase I and phase II clinical trial on patients having mCRPC patients and various advanced cancers (Chi et al. 2016) (Yu et al. 2018) (Rosenberg et al. 2018).

Their previous studies showed that Hsp27 drives CRPC progression by protecting its protein partners such as eIF4E and TCTP from ubiquitin-proteasome degradation (Andrieu et al. 2010) (Baylot et al. 2012). In order to have a larger overview of Hsp27-regulated proteins in PC, they conducted proteomic profiling of the LNCaP mock and LNCaP Hsp27 cells. This research illustrated that DDX5 expression is modulated by Hsp27. Additionally, their study on Hsp27 interactome by using IP/MS strategy which aimed to provide a bigger picture of Hsp27 partners revealed the interaction between Hsp27 and DDX5 in PC. Along with these findings, DDX5 originally captured their attention due to its well-known oncogenic roles in various human cancers, which made us believe that Hsp27 could confer CRPC through interaction with DDX5.

The DDX RNA helicase p68 (also called DDX5) plays crucial functions in pre-mRNA processing (Jalal et al. 2007) (Dardenne et al. 2012), mRNA decay (Bond et al. 2001), alternative splicing (Janknecht 2010), ribosome biogenesis and cell proliferation (Jalal et al. 2007), and transcriptional regulation (Fuller-Pace 2006). DDX5 has recently demonstrated to function in chromatin remodeling (Caretti et al. 2006), and to act as a transcriptional co-activator of difference transcription factors such as tumor suppressor p53 (Bates et al. 2005) (Nicol et al. 2013b), MyoD (Caretti et al. 2006), β-Catenin (Shin et al. 2007) (Clark et al. 2013) (Wang et al. 2015), Androgen Receptor AR (Clark et al. 2008). DDX5 has been shown to be over-expressed in nearly 93% of human cancers (Nyamao et al. 2019) including colorectal cancer (Shin et al. 2007) (Causevic et al. 2001), breast cancer (Mazurek et al. 2012) (Guturi et al. 2014), prostate cancer (Clark et al. 2008) (Clark et al. 2013), non-small-cell lung cancer (Wang et al. 2015), esophageal cancer (Ma et al. 2017). In addition, DDX5 has been demonstrated to enhance cell proliferation (Clark et al. 2008) (Ma et al. 2017) (Wang et al. 2015), metastasis (Yang et al. 2006), and resistance to drug (Cohen et al. 2008) through stimulating difference oncogenic signaling mechanisms (Yang et al. 2006) (Lin et al. 2013).

DDX5 overexpression is associated with the mCRPC progression, and it acts as an AR-coactivator which enhances transcriptional activity of AR and PSA (Prostate specific antigen) (Clark et al. 2008). In addition, DDX5 has been proved to interact with β-Catenin, a transcriptional co-regulator of AR and this interaction extremely intensifies the mediated AR transcription (Clark et al. 2013). Interestingly, DDX5 plays an important function in recruitment both AR and β-Catenin to the promotor of Androgen responsive genes and DDX5 also involves in elongation and transcriptional progress due to its physically direct binding to RNAPII (Clark et al. 2013). DDX5 has demonstrated to promote cell survival and growth in AR independent PC cell lines (DU 145, PC 3) by activating mTORC1 signaling (Taniguchi et al. 2016). Antisense oligonucleotides (ASO) have been demonstrated to be an advantageous tool in biology research and clinical application for years. Basically, ASOs which are short, synthetic singled—strand DNAs can bind to a specific RNA by base pairing and finally block protein translation by different mechanisms (Karaki et al. 2019). ASO-based strategies have been developing over 40 years and hold a great promising for treatment of different pathologies including cancer. In fact, a great number of ASO-mediated therapies have been tested in clinical treatment. There have been 8 ASOs so far which obtained FDA approval (US Food and Drug Administration) for Duchenne muscular dystrophy and spinal muscular atrophy therapy (Rinaldi and Wood 2018).

### SUMMARY OF THE INVENTION

In the present study, the inventors examined DDX5 protein expression in clinical relevance by using TMA. The results showed that elevated DDX5 is correlated with CRPC development, with aggressiveness and metastatic progression of the disease. To get a tool for our molecular mechanism study, and toward further clinic application, here they designed DDX5-targeting ASOs by a computer programmer and performed screenings to obtain an ASO that inhibits DDX5 protein efficiently. They determined two specific ASO (the hASO #51 and hmASO #3) which demonstrated to deplete DDX5 protein level in dose-dependency manner and inhibit cell proliferation significantly. Furthermore, they have conducted a study on DDX5 interactomes using IP/MS approach in order to get insight the functions of DDX5 during CRPC progression. The DDX5-associated proteins were identified in various PC cell lines ranging from the normal prostate cells, PNT1A (NM) to castration sensitive cells, LNCaP (CS) and castration resistance lines (CR) including DU145 and PC3. Functional analyses of the DDX5 interactome revealed that DDX5 could drive CRPC progression mainly via regulating various vital processes such as: DNA damage response, translation, transcription, RNA stability, and DNA conformation changes. They confirmed that DDX5 interacts with Ku70/Ku86, the core complex of Non homologous end-joining (NHEJ) pathway, and they proved that DDX5 negatively regulates DNA repair. Moreover, their current study showed that Hsp27 prevents DDX5 from proteasome degradation, and DDX5 regulates AKT/mTOR pathway via AKT and mTOR modulating. Besides showing the correlation between DDX5 and CRPC by TMA, their study revealed new functions of DDX5 in PC and provided better understanding about mechanism by which DDX5 promotes CRPC progression. Furthermore, they developed the first DDX5 inhibitor that can be used for combinational therapies in CRPC to restore treatment sensitivity.

The present invention relates to antisense oligonucleotides efficiency in reducing the expression of DDX5 in cancer of a subject. Particularly, the invention is defined by its claims.

### DETAILED DESCRIPTION OF THE INVENTION

### Sequences of the Invention

A first aspect of the invention relates to an inhibitor of DDX5 wherein said inhibitor reduces the expression and/or activity of DDX5 and targets the gene or the mRNA of DDX5.

In a particular embodiment, the invention relates to an inhibitor of DDX5 wherein said inhibitor reduces the expression and/or activity of DDX5 and targets the nucleic acids sequence SEQ ID NO: 94.

In a further embodiment, the invention relates to an inhibitor of DDX5 wherein said inhibitor reduces the expression and/or activity of DDX5 and targets a region comprising at least between 15 nucleic acids to 25 nucleic acids of SEQ ID NO: 94.

In a particular embodiment, the inhibitor targets at least, 15, 16, 17, 18, 19, 20, 21, 22, 23, 24 or 25 consecutive nucleic acids of SEQ ID NO: 94.

In one embodiment, the invention also relates to an inhibitor of DDX5 wherein said inhibitor reduces the expression and/or activity of DDX5 and targets at least the region comprising or consisting of the nucleic acids 276-515 of SEQ ID NO: 94.

In one embodiment, the invention also relates to an inhibitor of DDX5 wherein said inhibitor reduces the expression and/or activity of DDX5 and targets at least the region comprising or consisting of the nucleic acids 1056-1155 of SEQ ID NO: 94.

In one embodiment, the invention also relates to an inhibitor of DDX5 wherein said inhibitor reduces the expression and/or activity of DDX5 and targets at least the region comprising or consisting of the nucleic acids 1396-1795 of SEQ ID NO: 94.

In one embodiment, the invention also relates to an inhibitor of DDX5 wherein said inhibitor reduces the expression and/or activity of DDX5 and targets at least the region comprising or consisting of the nucleic acids 1856-1955 of SEQ ID NO: 94.

As used herein, the term “DDX5” for “Probable ATP-dependent RNA helicase DDX5” and also knows as “DEAD box protein 5” or “RNA helicase p68” refers to an RNA helicase. DDX5 is implicated in a number of cellular processes involving alteration of RNA secondary structure, such as translation initiation, nuclear and mitochondrial splicing, and ribosome and spliceosome assembly. Moreover, DDX5 has recently demonstrated to function in chromatin remodeling, and to act as a transcriptional co-activator of difference transcription factors such as tumor suppressor p53, MyoD, β-Catenin, Androgen Receptor AR.

The naturally occurring human DDX5 gene has a nucleotide sequence as shown in genebank accession number NC_000017.11 or Ensembl accession number DDX5 ENSG00000108654. According to the NCBI database, DDX5 has 4 major protein-coding mRNA sequences, which are NM_001320595.2, NM_004396.5, NM_001320596.2, NM_001320597.2, corresponding to the mRNA variant 1, variant 2, variant 3, and variant 4, respectively. The CDSs are strictly identical among 3 of 4 DDX5 transcript variants (encode the same isoform) and of 97% for the other one (variant 4) which has a distinct N terminus and is the same length as the main isoform. The reference sequence used to design ASOs was from the 1st transcript variant of DDX5 i.e. NM_001320595.2 and restricted to the CDS, which is defined as CCDS11659.1 (between nucleotides 256 and 2100 of SEQ ID NO: 94).

Thus, in a particular embodiment, the invention relates to an inhibitor of DDX5 wherein said inhibitor reduces the expression and/or activity of DDX5 and targets any variants 1, 2, 3 and/or 4 of DDX5.

In another particular embodiment, said inhibitor targets the nucleic acids sequence SEQ ID NO: 94 or SEQ ID NO: 95.

Thus, in a particular embodiment, the invention relates to an inhibitor of DDX5 wherein said inhibitor reduces the expression and/or activity of DDX5 and targets the nucleic acids sequence SEQ ID NO: 95.

In a further embodiment, the invention relates to an inhibitor of DDX5 wherein said inhibitor reduces the expression and/or activity of DDX5 and targets at least between 15 nucleic acids to 25 nucleic acids of SEQ ID NO: 95.

The nucleotide sequence cDNA of Homo sapiens DDX5 (variant 1) is defined by the sequence SEO ID NO: 94:

GACCATTCTC CGCCGACCAA AACCCGTCAA AGGATTATCA GACACGCGGG
TCGGACGGTC CACATCAGCC GGCAGCCCGG GCGGGTCCCG GGGTGCGAGC
AGCGCACTTC CGTAGTGCAG CTTCGGCTGG TGTCATCGGT GTCCTTCCTC
CGCTGCCGCC CCCGCAAGGC TTCGCCGTCA TCGAGGCCAT TTCCAGCGAC
TTGTCGCACG CTTTTCTATA TACTTCGTTC CCCGCCAACC GCAACCATTG
ACGCCATGTC GGGTTATTCG AGTGACCGAG ACCGCGGCCG GGACCGAGGG
TTTGGTGCAC CTCGATTTGG AGGAAGTAGG GCAGGGCCCT TATCTGGAAA
GAAGTTTGGA AACCCTGGGG AGAAATTAGT TAAAAAGAAG TGGAATCTTG
ATGAGCTGCC TAAATTTGAG AAGAATTTTT ATCAAGAGCA CCCTGATTTG
GCTAGGCGCA CAGCACAAGA GGTGGAAACA TACAGAAGAA GCAAGGAAAT
TACAGTTAGA GGTCACAACT GCCCGAAGCC AGTTCTAAAT TTTTATGAAG
CCAATTTCCC TGCAAATGTC ATGGATGTTA TTGCAAGACA GAATTTCACT
GAACCCACTG CTATTCAAGC TCAGGGATGG CCAGTTGCTC TAAGTGGATT
GGATATGGTT GGAGTGGCAC AGACTGGATC TGGGAAAACA TTGTCTTATT
TGCTTCCTGC CATTGTCCAC ATCAATCATC AGCCATTCCT AGAGAGAGGC
GATGGGCCTA TTTGTTTGGT GCTGGCACCA ACTCGGGAAC TGGCCCAACA
GGTGCAGCAA GTAGCTGCTG AATATTGTAG AGCATGTCGC TTGAAGTCTA
CTTGTATCTA CGGTGGTGCT CCTAAGGGAC CACAAATACG TGATTTGGAG
AGAGGTGTGG AAATCTGTAT TGCAACACCT GGAAGACTGA TTGACTTTTT
AGAGTGTGGA AAAACCAATC TGAGAAGAAC AACCTACCTT GTCCTTGATG
AAGCAGATAG AATGCTTGAT ATGGGCTTTG AACCCCAAAT AAGGAAGATT
GTGGATCAAA TAAGACCTGA TAGGCAAACT CTAATGTGGA GTGCGACTTG
GCCAAAAGAA GTAAGACAGC TTGCTGAAGA TTTCCTGAAA GACTATATTC
ATATAAACAT TGGTGCACTT GAACTGAGTG CAAACCACAA CATTCTTCAG
ATTGTGGATG TGTGTCATGA CGTAGAAAAG GATGAAAAAC TTATTCGTCT
AATGGAAGAG ATCATGAGTG AGAAGGAGAA TAAAACCATT GTTTTTGTGG
AAACCAAAAG AAGATGTGAT GAGCTTACCA GAAAAATGAG GAGAGATGGG
TGGCCTGCCA TGGGTATCCA TGGTGACAAG AGTCAACAAG AGCGTGACTG
GGTTCTAAAT GAATTCAAAC ATGGAAAAGC TCCTATTCTG ATTGCTACAG
ATGTGGCCTC CAGAGGGCTA GATGTGGAAG ATGTGAAATT TGTCATCAAT
TATGACTACC CTAACTCCTC AGAGGATTAT ATTCATCGAA TTGGAAGAAC
TGCTCGCAGT ACCAAAACAG GCACAGCATA CACTTTCTTT ACACCTAATA
ACATAAAGCA AGTGAGCGAC CTTATCTCTG TGCTTCGTGA AGCTAATCAA
GCAATTAATC CCAAGTTGCT TCAGTTGGTC GAAGACAGAG GTTCAGGTCG
TTCCAGGGGT AGAGGAGGCA TGAAGGATGA CCGTCGGGAC AGATACTCTG
CGGGCAAAAG GGGTGGATTT AATACCTTTA GAGACAGGGA AAATTATGAC
AGAGGTTACT CTAGCCTGCT TAAAAGAGAT TTTGGGGCAA AAACTCAGAA
TGGTGTTTAC AGTGCTGCAA ATTACACCAA TGGGAGCTTT GGAAGTAATT
TTGTGTCTGC TGGTATACAG ACCAGTTTTA GGACTGGTAA TCCAACAGGG
ACTTACCAGA ATGGTTATGA TAGCACTCAG CAATACGGAA GTAATGTTCC
AAATATGCAC AATGGTATGA ACCAACAGGC ATATGCATAT CCTGCTACTG
CAGCTGCACC TATGATTGGT TATCCAATGC CAACAGGATA TTCCCAATAA
GACTTTAGAA GTATATGTAA ATGTCTGTTT TTCATAATTG ctctttatat
TGTGTGTTAT CTGACAAGAT AGTTATTTAA GAAACATGGG AATTGCAGAA
ATGACTGCAG TGCAGCAGTA ATTATGGTGC ACTTTTTCGC TATTTAAGTT
GGATATTTCT CTACATTCCT GAAACAATTT TTAGGTTTTT TTTGTACTAG
AAAATGCAGG CAGTGTTTTC ACAAAAGTAA ATGTACAGTG ATTTGAAATA
CAATAAATGA AGGCAATGCA TGGCCTTCCA ATAAAAAATA TTTGAAGACT
GAATTAAGTG GAAATTGTAC TTTATTTTAT ATAATGTCAT GTAAAACTTT
GCTTAAGATG GTCTGGTTTT TTTTTTGTTT TTGTTTGGTT TTTTTTTTCC
ATGAAAACAA ATGACTGTTC CTTTTTATTT AATTTGGGAG GCAGGGGGAA
TCAGAAGGCC CTTCTTTATA ATGAGCTATT CATATTGCAG GAGTCAGAAT
GAATTGATAC AGGTGAATTT TTAGTTACAG GCTAAATTGC ATAAAAGCTT
TGTCAGCTTC CAGCATCAGG GGAGTCATTT AATAGCCTTT TTCCTTATTT
GCTAGTATGG TTAAATGAGA AAATAGTAAA ATAGATACAA AGTCATCTAT
ATAGTGTGAG AACGTGGGTG actttttcaa AGTTTATAAT TTAAAAAGCT
CCAAATAACT GGCTTTTTCA AGAGACTTAT ACTCATGCTC TTGGCTATAC
TGTGAATTAC TGAAATGTTG AACAAACCTG TGAAAGACAT ACATTAGCCC
TTTAAGATGG CCAGGAGCTA AGCTTGAGTC TCCTTTACTG AATTTCGTTC
TTAGTGCAGG TTACTTGTAG ATTCTAGTCT TCACAGGCTC CCTGGGGCTC
TTAACTAGTC ACACTGGGAG TCATGAATGT CTTTCCAATA ATTCAGGGAA
TTCTAGAGAT CCTCAAACTG TAAGGTCTAT TCATACTCAA CACAAGGAAA
AAACCTCATT AAAATTAATG ACTAATCAGG AGGCAACGTA ACCAAAAGCA
CAGTGAATGA AAGTTTTCAT GGTAGGTTCA ACATGGGTTT ATTGCTAGAA
AGATCCAGGG GATAGCTTTA GGTTTAACTT CGGCTCACCA ACGTAACTTT
CTAATCATTT ATTTCAGTAA TAGCTAGAAG TGGGTCTGAA TGTTTTCCCA
GAGTCTGATA CGTGTTTTTT TTTGCCAGAA GAGAGGTCTT CAGGAGACTT
CATTTAAATT CTGATTATTA AACTGAGGCT TTAATTGATG TTAATGCCTT
ATGTCAAATG TAAAGTTAGA ATTTGCTAGG GCTGGGATAG GGAGTGATAT
TTCTAGGACT TAGACATTGA AAACTAATTC AGCCTGTAGT AACCTGGATG
GTTTTCAATG GCATGGTTAG TCAAATTCAT GGTTTTAAAC TTAGAAGCAG
CTTTCGGGGG AGAGGGTAGG TTGGAGCATT TATTACATAT TTTACTGTTT
AATGTCTTAA CCGTGGGCCT TTTAATTTGT AAACACTGAA ATGATTGTTG
GGCTGTGGAA AACATTTACC TATTTACCTT GGAAGTTTTA AAAGACAGTC
CACTTTTTAG CATGTGTGTT GTGTCCAGCC TGTGGTCGTC TTAACTAATA
AATGTGATTT TTCTCCCCA

The nucleotide sequence cDNA of Homo sapiens DDX5 (variant 4) is defined by the sequence SEQ ID NO: 95:

GCCATTTTGATATTCACGTCACAGTGATTGGAAGAGATTTGACGGTGTAGT
GTCTTCAAGCTTGCTTTTTGTGTGGGGATTGGGGAGCTGTCGGGGCGGCTGCCATT
TGGTAGCTGTTGAGGGAGTTGAGAGGGAGCGTATTGTGCGGATGAAAGCGGGAC
GCTTCGAGGCAGACGAAGGAACATCTGTTAGGTTTGGTGCACCTCGATTTGGAGG
AAGTAGGGCAGGGCCCTTATCTGGAAAGAAGTTTGGAAACCCTGGGGAGAAATT
AGTTAAAAAGAAGTGGAATCTTGATGAGCTGCCTAAATTTGAGAAGAATTTTTAT
CAAGAGCACCCTGATTTGGCTAGGCGCACAGCACAAGAGGTGGAAACATACAGA
AGAAGCAAGGAAATTACAGTTAGAGGTCACAACTGCCCGAAGCCAGTTCTAAAT
TTTTATGAAGCCAATTTCCCTGCAAATGTCATGGATGTTATTGCAAGACAGAATTT
CACTGAACCCACTGCTATTCAAGCTCAGGGATGGCCAGTTGCTCTAAGTGGATTG
GATATGGTTGGAGTGGCACAGACTGGATCTGGGAAAACATTGTCTTATTTGCTTC
CTGCCATTGTCCACATCAATCATCAGCCATTCCTAGAGAGAGGCGATGGGCCTAT
TTGTTTGGTGCTGGCACCAACTCGGGAACTGGCCCAACAGGTGCAGCAAGTAGCT
GCTGAATATTGTAGAGCATGTCGCTTGAAGTCTACTTGTATCTACGGTGGTGCTCC
TAAGGGACCACAAATACGTGATTTGGAGAGAGGTGTGGAAATCTGTATTGCAAC
ACCTGGAAGACTGATTGACTTTTTAGAGTGTGGAAAAACCAATCTGAGAAGAAC
AACCTACCTTGTCCTTGATGAAGCAGATAGAATGCTTGATATGGGCTTTGAACCC
CAAATAAGGAAGATTGTGGATCAAATAAGACCTGATAGGCAAACTCTAATGTGG
AGTGCGACTTGGCCAAAAGAAGTAAGACAGCTTGCTGAAGATTTCCTGAAAGAC
TATATTCATATAAACATTGGTGCACTTGAACTGAGTGCAAACCACAACATTCTTC
AGATTGTGGATGTGTGTCATGACGTAGAAAAGGATGAAAAACTTATTCGTCTAAT
GGAAGAGATCATGAGTGAGAAGGAGAATAAAACCATTGTTTTTGTGGAAACCAA
AAGAAGATGTGATGAGCTTACCAGAAAAATGAGGAGAGATGGGTGGCCTGCCAT
GGGTATCCATGGTGACAAGAGTCAACAAGAGCGTGACTGGGTTCTAAATGAATTC
AAACATGGAAAAGCTCCTATTCTGATTGCTACAGATGTGGCCTCCAGAGGGCTAG
ATGTGGAAGATGTGAAATTTGTCATCAATTATGACTACCCTAACTCCTCAGAGGA
TTATATTCATCGAATTGGAAGAACTGCTCGCAGTACCAAAACAGGCACAGCATAC
ACTTTCTTTACACCTAATAACATAAAGCAAGTGAGCGACCTTATCTCTGTGCTTCG
TGAAGCTAATCAAGCAATTAATCCCAAGTTGCTTCAGTTGGTCGAAGACAGAGGT
TCAGGTCGTTCCAGGGGTAGAGGAGGCATGAAGGATGACCGTCGGGACAGATAC
TCTGCGGGCAAAAGGGGTGGATTTAATACCTTTAGAGACAGGGAAAATTATGAC
AGAGGTTACTCTAGCCTGCTTAAAAGAGATTTTGGGGCAAAAACTCAGAATGGTG
TTTACAGTGCTGCAAATTACACCAATGGGAGCTTTGGAAGTAATTTTGTGTCTGCT
GGTATACAGACCAGTTTTAGGACTGGTAATCCAACAGGGACTTACCAGAATGGTT
ATGATAGCACTCAGCAATACGGAAGTAATGTTCCAAATATGCACAATGGTATGAA
CCAACAGGCATATGCATATCCTGCTACTGCAGCTGCACCTATGATTGGTTATCCA
ATGCCAACAGGATATTCCCAATAAGACTTTAGAAGTATATGTAAATGTCTGTTTTT
CATAATTGCTCTTTATATTGTGTGTTATCTGACAAGATAGTTATTTAAGAAACATG
GGAATTGCAGAAATGACTGCAGTGCAGCAGTAATTATGGTGCACTTTTTCGCTAT
TTAAGTTGGATATTTCTCTACATTCCTGAAACAATTTTTAGGTTTTTTTTGTACTAG
AAAATGCAGGCAGTGTTTTCACAAAAGTAAATGTACAGTGATTTGAAATACAATA
AATGAAGGCAATGCATGGCCTTCCAATAAAAAATATTTGAAGACTGAATTAAGTG
GAAATTGTACTTTATTTTATATAATGTCATGTAAAACTTTGCTTAAGATGGTCTGG
TTTTTTTTTTGTTTTTGTTTGGTTTTTTTTTTCCATGAAAACAAATGACTGTTCCTTT
TTATTTAATTTGGGAGGCAGGGGGAATCAGAAGGCCCTTCTTTATAATGAGCTAT
TCATATTGCAGGAGTCAGAATGAATTGATACAGGTGAATTTTTAGTTACAGGCTA
AATTGCATAAAAGCTTTGTCAGCTTCCAGCATCAGGGGAGTCATTTAATAGCCTT
TTTCCTTATTTGCTAGTATGGTTAAATGAGAAAATAGTAAAATAGATACAAAGTC
ATCTATATAGTGTGAGAACGTGGGTGACTTTTTCAAAGTTTATAATTTAAAAAGC
TCCAAATAACTGGCTTTTTCAAGAGACTTATACTCATGCTCTTGGCTATACTGTGA
ATTACTGAAATGTTGAACAAACCTGTGAAAGACATACATTAGCCCTTTAAGATGG
CCAGGAGCTAAGCTTGAGTCTCCTTTACTGAATTTCGTTCTTAGTGCAGGTTACTT
GTAGATTCTAGTCTTCACAGGCTCCCTGGGGCTCTTAACTAGTCACACTGGGAGT
CATGAATGTCTTTCCAATAATTCAGGGAATTCTAGAGATCCTCAAACTGTAAGGT
CTATTCATACTCAACACAAGGAAAAAACCTCATTAAAATTAATGACTAATCAGGA
GGCAACGTAACCAAAAGCACAGTGAATGAAAGTTTTCATGGTAGGTTCAACATG
GGTTTATTGCTAGAAAGATCCAGGGGATAGCTTTAGGTTTAACTTCGGCTCACCA
ACGTAACTTTCTAATCATTTATTTCAGTAATAGCTAGAAGTGGGTCTGAATGTTTT
CCCAGAGTCTGATACGTGTTTTTTTTTGCCAGAAGAGAGGTCTTCAGGAGACTTC
ATTTAAATTCTGATTATTAAACTGAGGCTTTAATTGATGTTAATGCCTTATGTCAA
ATGTAAAGTTAGAATTTGCTAGGGCTGGGATAGGGAGTGATATTTCTAGGACTTA
GACATTGAAAACTAATTCAGCCTGTAGTAACCTGGATGGTTTTCAATGGCATGGT
TAGTCAAATTCATGGTTTTAAACTTAGAAGCAGCTTTCGGGGGAGAGGGTAGGTT
GGAGCATTTATTACATATTTTACTGTTTAATGTCTTAACCGTGGGCCTTTTAATTT
GTAAACACTGAAATGATTGTTGGGCTGTGGAAAACATTTACCTATTTACCTTGGA
AGTTTTAAAAGACAGTCCACTTTTTAGCATGTGTGTTGTGTCCAGCCTGTGGTCGT
CTTAACTAATAAATGTGATTTTTCTCCCCA

As used herein, the term “inhibitor” refers to a natural or synthetic compound that has a biological effect to inhibit or reduce the expression and/or activity of DDX5.

In a particular embodiment, the inhibitor of the invention refers to a natural or synthetic compound that has a biological effect to reduce and/or inhibit the expression of DDX5 gene. It will be understood to the skilled in the art that inhibiting expression of a gene, e.g. the DDX5 gene, typically results in a decrease or even abolition of the gene product (ARN and/or protein, e.g. DDX5 protein) in target cells or tissues, although various levels of inhibition may be achieved. Inhibiting or decreasing expression is typically referred to as knockdown.

In a particular embodiment, the inhibitor of activity of DDX5 refers to a natural or synthetic compound that has a biological effect to reduce and/or inhibit the activity of DDX5.

In a particular embodiment, the inhibitor of DDX5 of the invention is a siRNA, a shRNA, an antisense oligonucleotide, miRNA or a ribozyme.

In one embodiment, the inhibitor of DDX5 according to the invention is a siRNA.

Small inhibitory RNAs, also referred to as short interfering RNAs (siRNAs) can also function as DDX5 expression inhibitors for use in the present invention. DDX5 activity can be reduced by treating the subject or cell with a small double stranded RNA (dsRNA), or a vector or construct causing the production of a small double stranded RNA, such that DDX5 expression is specifically inhibited (i.e. RNA interference or RNAi) by degradation of mRNAs in a sequence specific manner. Methods for selecting an appropriate dsRNA or dsRNA-encoding vector are known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836, each of which is incorporated by reference herein in its entirety).

In a particular embodiment, the invention relates to an inhibitor of DDX5 wherein said inhibitor is siRNA.

In a particular embodiment, the siRNA according to the invention targets the region comprising or consisting the nucleic acids 276-515, 1056-1155, 1396-1795 or 1856-1955 of SEQ ID NO: 94.

In one embodiment, the inhibitor of DDX5 according to the invention is a shRNA.

Short hairpin RNAs (shRNA) can also function as DDX5 expression inhibitors for use in the present invention. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound.

In one embodiment, the inhibitor of DDX5 according to the invention is a miRNA. miRNAs (miR) can also function as DDX5 expression inhibitors for use in the present invention. miRNA has its general meaning in the art and refers to microRNA molecules that are generally 21 to 22 nucleotides in length, even though lengths of 19 and up to 23 nucleotides have been reported, and suppress translation of targeted mRNAs. miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem. The processed miRNA (also referred to as “mature miRNA”) becomes part of a large complex to downregulate, e.g. decrease translation, of a particular target gene.

Multiple miRNAs may be employed to knockdown DDX5. The miRNAs may be complementary to different target transcripts or different binding sites of a target transcript. Polycistronic transcripts may also be utilized to enhance the efficiency of target gene knockdown. In some embodiments, multiple genes encoding the same miRNAs or different miRNAs may be regulated together in a single transcript, or as separate transcripts in a single vector cassette. In one embodiment, the vector is a viral vector, including but not limited to recombinant adeno-associated viral (rAAV) vectors, lentiviral vectors, retroviral vectors and retrotransposon-based vector systems.

In one embodiment, the inhibitor of DDX5 is an antisense nucleic acid.

Iinhibitor of DDX5 expression of the invention is based on antisense oligonucleotide constructs. Anti-sense oligonucleotides, including antisense RNA molecules and antisense DNA molecules, would act to directly block the translation of DDX5 mRNA by binding thereto and thus preventing protein translation or by increasing mRNA degradation, thus decreasing the level of DDX5 proteins, and thus activity, in a cell. For example, antisense oligonucleotides of at least about 15 bases and complementary to unique regions of the mRNA transcript sequence encoding DDX5 can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732, each of which is incorporated by reference herein in its entirety).

The antisense RNA that is complementary to the sense target sequence is encoded by a DNA sequence for the production of any of the foregoing inhibitors (e.g., antisense, siRNAs, shRNAs or miRNAs). The DNA encoding double stranded RNA of interest is incorporated into a gene cassette, e.g. an expression cassette in which transcription of the DNA is controlled by a promoter.

In a particular embodiment, the inhibitor of DDX5 gene expression is an antisense oligonucleotide.

In a particular embodiment, the inhibitor of DDX5 gene expression is an isolated, synthetic or recombinant antisense oligonucleotide targeting the DDX5 mRNA transcript. The oligonucleotide of the invention can be of any suitable type.

In some embodiments, the oligonucleotide is a RNA oligonucleotide. In some embodiments, the oligonucleotide is a DNA oligonucleotide.

Thus, the invention also relates to an antisense oligonucleotide which reduces the expression and/or activity of DDX5 and targets the gene or the mRNA of DDX5.

In a particular embodiment, the antisense oligonucleotide of the invention reduces the expression and/or activity of DDX5 and targets the nucleotides sequence SEQ ID NO: 94.

In a further embodiment, the antisense oligonucleotide of the invention reduces the expression and/or activity of DDX5 and targets at least between 15 nucleic acids to 25 nucleic acids of SEQ ID NO: 94.

In a particular embodiment, the antisense oligonucleotide targets at least, 15, 16, 17, 18, 19, 20, 21, 22, 23, 24 or 25 consecutive nucleic acids of SEQ ID NO: 94.

In one embodiment, the antisense oligonucleotide of the invention reduces the expression and/or activity of DDX5 and targets at least the region comprising or consisting of the nucleic acids 276-515 of SEQ ID NO: 94.

In one embodiment, the antisense oligonucleotide of the invention reduces the expression and/or activity of DDX5 and targets at least the region comprising or consisting of the nucleic acids 1056-1155 of SEQ ID NO: 94.

In one embodiment, the antisense oligonucleotide of the invention reduces the expression and/or activity of DDX5 and targets at least the region comprising or consisting of the nucleic acids 1396-1795 of SEQ ID NO: 94.

In one embodiment, the antisense oligonucleotide of the invention reduces the expression and/or activity of DDX5 and targets at least the region comprising or consisting of the nucleic acids 1856-1955 of SEQ ID NO: 94.

In some embodiments, the antisense oligonucleotide of the present invention has a length of at least 15 nucleic acids.

In some embodiments, the antisense oligonucleotide of the present invention has a length from 15 to 25 nucleic acids.

In particular, the antisense oligonucleotide of the present invention has a length of 15, 16, 17, 18, 19, 20, 21, 22, 23, 24 or 25 nucleic acids.

Particularly, the antisense oligonucleotide of the present invention has a length of 20 nucleic acids.

In a particular embodiment, the antisense oligonucleotide is selected from the group consisting of but not limited to: SEQ ID NO: 1 to SEQ ID NO:93 (see Table 1).

TABLE 1
Sequences of antisense oligonucleotides useful for
their capacity to reduce the expression and/or
activity of DDX5

| | | | | SEQ |
|---|---|---|---|---|
| | | | | ID |
| No ASO | Position | Antisens 5′-3′ | % CG | NO: |
| 1 | 256-275 | tcactcgaataacccgacat | 45 | 1 |
| 2 | 276-295 | ggtcccggccgcggtctcgg | 85 | 2 |
| 3 | 296-315 | tcgcggtgcaccaaaccctc | 65 | 3 |
| 4 | 316-335 | cctgccctacttcctccaaa | 55 | 4 |
| 5 | 336-355 | acttctttccagataagggc | 45 | 5 |
| 6 | 356-375 | tttctccccagggtttccaa | 50 | 6 |
| 7 | 376-395 | ttccacttctttttaactaa | 25 | 7 |
| 8 | 396-415 | atttaggcagctcatcaaga | 40 | 8 |
| 9 | 416-435 | ttgataaaattcttctcaa | 20 | 9 |
| 10 | 436-455 | ctagccaaatcagggtgctc | 55 | 10 |
| 11 | 456-475 | ccacctcttgtgctgtgcgc | 60 | 11 |
| 12 | 476-495 | cttgcttcttctgtatgttt | 35 | 12 |
| 13 | 496-515 | tgacctctaactgtaatttc | 35 | 13 |
| 14 | 516-535 | gaactggcttcgggcagttg | 60 | 14 |
| 15 | 536-555 | attggcttcataaaaattta | 20 | 15 |
| 16 | 556-575 | tccatgacatttgcagggaa | 45 | 16 |
| 17 | 576-595 | aattctgtcttgcaataaca | 30 | 17 |
| 18 | 596-615 | aatacgagtgggttcagtga | 45 | 18 |
| 19 | 616-635 | actggccatccctgtgcttg | 60 | 19 |
| 20 | 636-655 | tatccaatcccacttagagca | 45 | 20 |
| 21 | 656-675 | agtctgtgccactccaacca | 55 | 21 |
| 22 | 676-695 | gacaatgttttcccagatcc | 40 | 22 |
| 23 | 696-715 | caatggcaggaagcaaataa | 40 | 23 |
| 24 | 716-735 | tggctgatgattgatgtgga | 45 | 24 |
| 25 | 736-755 | ccatcgcctctctctaggaa | 55 | 25 |
| 26 | 756-775 | ccagcaccaaacaaataggc | 50 | 26 |
| 27 | 776-795 | ggccagttcccgagttggtg | 65 | 27 |
| 28 | 796-815 | gctacttgctgcacctgttg | 55 | 28 |
| 29 | 816-835 | atgctctacaatattcacga | 35 | 29 |
| 30 | 836-855 | acaagtagattcaagcgac | 40 | 30 |
| 31 | 856-875 | ttaggagcaccaccgtagat | 50 | 31 |
| 32 | 876-895 | aatcacgtatttgtggtccc | 45 | 32 |
| 33 | 896-915 | gatttcctcattctctcca | 40 | 33 |
| 34 | 916-935 | cttccaggtgttgcaataca | 45 | 34 |
| 35 | 936-955 | actctaaaaagtcaatcagt | 30 | 35 |
| 36 | 956-975 | tctcagattggtttttccac | 40 | 36 |
| 37 | 976-995 | aggacaaggtaggttgttct | 45 | 37 |
| 38 | 996-1015 | gcattctatctgcttcatca | 40 | 38 |
| 39 | 1016-1035 | gggttcaaagcccatatcaa | 45 | 39 |
| 40 | 1036-1055 | tccacaatcttccttatttg | 35 | 40 |
| 41 | 1056-1075 | gcctatcaggtcttatttga | 40 | 41 |
| 42 | 1076-1095 | cgcactccacattagagttt | 45 | 42 |
| 43 | 1096-1115 | cttacttctttcggccaagt | 45 | 43 |
| 44 | 1116-1135 | ggaaatcttcagcaagctgt | 45 | 44 |
| 45 | 1136-1155 | tatatgaatatagtctttca | 20 | 45 |
| 46 | 1156-1175 | agttcaagtgcaccaatgtt | 40 | 46 |
| 47 | 1176-1195 | gaatgttgtggtttgcactc | 45 | 47 |
| 48 | 1196-1215 | acacacatccacaatgtgaa | 40 | 48 |
| 49 | 1216-1235 | tcatccttttctacgtcatg | 40 | 49 |
| 50 | 1236-1255 | ccattagacgaataagtttt | 60 | 50 |
| 51 | 1256-1275 | cttctcactcatgatctctt | 40 | 51 |
| 52 | 1276-1295 | aaaacaatggttttattctc | 25 | 52 |
| 53 | 1296-1315 | atcttcttttggtttccaca | 35 | 53 |
| 54 | 1316-1335 | ttttgtggtagctcatcac | 40 | 54 |
| 55 | 1336-1355 | ggccacccatctctcctcat | 60 | 55 |
| 56 | 1356-1375 | caccatggatacccatggca | 55 | 56 |
| 57 | 1376-1395 | acgctcttgttgactcttgt | 45 | 57 |
| 58 | 1396-1415 | aattcatttagaaccctgtc | 35 | 58 |
| 59 | 1416-1435 | taggagcttttccatgtttg | 40 | 59 |
| 60 | 1436-1455 | cacatctgtagcaatcagaa | 40 | 60 |
| 61 | 1456-1475 | acatctagccctctggaggc | 60 | 61 |
| 62 | 1476-1495 | tgacaaatttcacatcttcc | 35 | 62 |
| 63 | 1496-1515 | gttagggtagtcataattga | 35 | 63 |
| 64 | 1516-1535 | tgaatataatcctctgagga | 35 | 64 |
| 65 | 1536-1555 | gagcagttcttccaattcga | 45 | 65 |
| 66 | 1556-1575 | tgtgcctgttttggtactgc | 50 | 66 |
| 67 | 1576-1595 | ggtgtaaagaaagtgtatgc | 35 | 67 |
| 68 | 1596-1615 | tcacttgctttatgttatta | 25 | 68 |
| 69 | 1616-1635 | aagcacagagataaggtcgc | 50 | 69 |
| 70 | 1636-1655 | attgcttgattagcttcacg | 40 | 70 |
| 71 | 1656-1675 | actgaagcaacttgggatta | 40 | 71 |
| 72 | 1676-1695 | tgaacctctgtcttcgacca | 50 | 72 |
| 73 | 1696-1715 | cctctacccctggaacgacc | 65 | 73 |
| 74 | 1716-1735 | gacggtcatccttcatgcct | 55 | 74 |
| 75 | 1736-1755 | gcccgcagagtatctgtccc | 65 | 75 |
| 76 | 1756-1775 | gtattaaatccacccctttt | 35 | 76 |
| 77 | 1776-1795 | aattttccctgtctctaaag | 35 | 77 |
| 78 | 1796-1815 | gctagagtaacctctgtcat | 45 | 78 |
| 79 | 1816-1835 | ccaaaatctcttttaagcag | 35 | 79 |
| 80 | 1836-1855 | caccattctgagtttttgcc | 45 | 80 |
| 81 | 1856-1875 | gtaatttgcagcatgtaaa | 30 | 81 |
| 82 | 1876-1895 | cttccaaagctcccattggt | 50 | 82 |
| 83 | 1896-1915 | taccagcagacacaaaatta | 35 | 83 |
| 84 | 1916-1935 | agtcctaaaactggtctgta | 40 | 84 |
| 85 | 1936-1955 | taagtccctgttggattacc | 45 | 85 |
| 86 | 1956-1975 | tgctatcataaccattctgg | 40 | 86 |
| 87 | 1976-1995 | attacttccgtattgctgag | 40 | 87 |
| 88 | 1996-2015 | ccattgtgcatatttggaac | 40 | 88 |
| 89 | 2016-2035 | catatgcctgttggttcata | 40 | 89 |
| 90 | 2036-2055 | agctgcagtagcaggatatg | 50 | 90 |
| 91 | 2056-2075 | ggataaccaatgataggtgc | 45 | 91 |
| 92 | 2076-2095 | gggaatatcctgttggcatt | 45 | 92 |
| 93 | 2096-2115 | tatacttctaaagtcttatt | 20 | 93 |

In a particular embodiment, the antisense oligonucleotide is selected from the group consisting of but not limited to: SEQ ID NO: 1 to SEQ ID NO:50, and SEQ ID NO:52 to SEQ ID NO:93 (see Table 1).

In a particular embodiment, the antisense oligonucleotide is set forth as SEQ ID NO:3.

As used herein, the term “acid nucleic” or “nucleotide” is defined as a modified or naturally occurring deoxyribonucleotide or ribonucleotide. Nucleotides typically include purines and pyrimidines, which include thymidine (T), cytidine (C), guanosine (G), adenosine (A) and uridine (U).

As used herein, the term “oligonucleotide” refers to an oligomer of the nucleotides defined above. The term “oligonucleotide” refers to a nucleic acid sequence, 3′-5′ or 5′-3′ oriented, which may be single- or double-stranded. The oligonucleotide used in the context of the invention may in particular be DNA or RNA. The term also includes “oligonucleotide analog” which refers to an oligonucleotide having (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide {e.g., single-stranded RNA or single-stranded DNA). Particularly, analogs are those having a substantially uncharged, phosphorus containing backbone. A substantially uncharged, phosphorus containing backbone in an oligonucleotide analog is one in which a majority of the subunit linkages, e.g., between 50-100%, typically at least 60% to 100% or 75% or 80% of its linkages, are uncharged, and contain a single phosphorous atom.

The term “oligonucleotide” also refers to an oligonucleotide sequence that is inverted relative to its normal orientation for transcription and so correspond to a RNA or DNA sequence that is complementary to a target gene mRNA molecule expressed within the host cell (e.g., it can hybridize to the target gene mRNA molecule through Watson-Crick base pairing).

An antisense strand can be constructed in a number of different ways, provided that it is capable of interfering with the expression of a target gene. For example, the antisense strand can be constructed by reverse-complementing the coding region (or a portion thereof) of the target gene relative to its normal orientation for transcription to allow the transcription of its complement, (e.g., RNAs encoded by the antisense and sense gene may be complementary). In some embodiments, the oligonucleotide need not have the same intron or exon pattern as the target gene, and noncoding segments of the target gene may be equally effective in achieving antisense suppression of target gene expression as coding segments such as antisense oligonucleotide (ASO). In some embodiments, the oligonucleotide has the same exon pattern as the target gene such as siRNA and antisense oligonucleotide (ASO).

As used herein, the term “target” or “targeting” refers to an oligonucleotide able to specifically bind to a FYXD2 gene or a DDX5 mRNA (any variants of the mRNA) encoding a DDX5 gene product. In particular, it refers to an oligonucleotide able to inhibit said gene or said mRNA by the methods known to the skilled in the art (e.g. antisense, RNA interference).

According to the invention, the antisense oligonucleotide of the present invention targets an mRNA and/or DNA encoding DDX5 gene product, and is capable of reducing the amount of DDX5 expression and/or activity in cells.

That is to say, the antisense oligonucleotide comprises a sequence that is at least partially complementary, particularly perfectly complementary, to a region of the sequence of said mRNA, said complementarity being sufficient to yield specific binding under intra-cellular conditions. As immediately apparent to the skilled in the art, by a sequence that is “perfectly complementary to” a second sequence is meant the reverse complement counterpart of the second sequence, either under the form of a DNA molecule or under the form of a RNA molecule. A sequence is “partially complementary to” a second sequence if there are one or more mismatches.

The antisense oligonucleotide of the present invention that targets a cDNA or mRNA encoding DDX5 gene can be designed by using the sequence of said mRNA as a basis, e.g. using bioinformatic tools.

Particularly, the antisense oligonucleotide according to the invention is capable of reducing the expression and/or activity of DDX5 in cancer cells. Methods for determining whether an oligonucleotide is capable of reducing the expression and/or activity of DDX5 in cells are known to those skilled in the art.

This can be performed for example be done by analyzing DDX5 RNA expression such as by RT-qPCR, in situ hybridization or DDX5 protein expression such as by immunohistochemistry, Western blot, and by comparing DDX5 protein expression or DDX5 functional activity in the presence and in the absence of the antisense oligonucleotide to be tested.

In other embodiments, the oligonucleotide is targeted to a translation initiation site (AUG codon), sequences in the coding region (e.g. one or more exons), 5′-untranslated region or 3′-untranslated region of an mRNA. The aim is to interfere with functions of the messenger RNA include all vital functions including translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with protein expression.

In some embodiments, the oligonucleotide of the present invention is further modified, particularly chemically modified, in order to increase the stability and/or therapeutic efficiency in vivo. The one skilled in the art can easily provide some modifications that will improve the efficacy of the oligonucleotide such as stabilizing modifications (C. Frank Bennett and Eric E. Swayze, RNA Targeting Therapeutics: Molecular Mechanisms of Antisense Oligonucleotides as a Therapeutic Platform Annu. Rev. Pharmacol. Toxicol. 2010.50:259-293; Juliano R L. The delivery of therapeutic oligonucleotides. Nucleic Acids Res. 2016 Aug. 19; 44(14):6518-48). In particular, the oligonucleotide used in the context of the invention may comprise modified nucleotides. Chemical modifications may occur at three different sites: (i) at phosphate groups, (ii) on the sugar moiety, and/or (iii) on the entire backbone structure of the oligonucleotide. Typically, chemical modifications include backbone modifications, heterocycle modifications, sugar modifications, and conjugation strategies.

For example the oligonucleotide is be selected from the group consisting of oligodeoxyribonucleotides, oligoribonucleotides, small regulatory RNAs (sRNAs), U7- or U1-mediated ASOs or conjugate products thereof such as peptide-conjugated or nanoparticle-complexed ASOs, chemically modified oligonucleotide by backbone modifications such as morpholinos, phosphorodiamidate morpholino oligomers (Phosphorodiamidate morpholinos, PMO), peptide nucleic acid (PNA), phosphorothioate (PS) oligonucleotides, stereochemically pure phosphorothioate (PS) oligonucleotides, phosphoramidates modified oligonucleotides, thiophosphoramidate-modified oligonucleotides, and methylphosphonate modified oligonucleotides; chemically modified oligonucleotide by heterocycle modifications such as bicycle modified oligonucleotides, Bicyclic Nucleic Acid (BNA), tricycle modified oligonucleotides, tricyclo-DNA-antisense oligonucleotides (ASOs), nucleobase modifications such as 5-methyl substitution on pyrimidine nucleobases, 5-substituted pyrimidine analogues, 2-Thio-thymine modified oligonucleotides, and purine modified oligonucleotides; chemically modified oligonucleotide by sugar modifications such as Locked Nucleic Acid (LNA) oligonucleotides, 2′,4′-Methyleneoxy Bridged Nucleic Acid (BNA), ethylene-bridged nucleic acid (ENA), constrained ethyl (cEt) oligonucleotides, 2′-Modified RNA, 2′- and 4′-modified oligonucleotides such as 2′-O-Me RNA (2′-OMe), 2′-O-Methoxyethyl RNA (MOE), 2′-Fluoro RNA (FRNA), and 4′-Thio-Modified DNA and RNA; chemically modified oligonucleotide by conjugation strategies such as N-acetyl galactosamine (GalNAc) oligonucleotide conjugates such as 5′-GalNAc and 3′-GalNAc ASO conjugates, lipid oligonucleotide conjugates (LASO), cell penetrating peptides (CPP) oligonucleotide conjugates, targeted oligonucleotide conjugates, antibody-oligonucleotide conjugates, polymer-oligonucleotide conjugate such as with PEGylation and targeting ligand; and chemical modifications and conjugation strategies described for example in Bennett and Swayze, 2010 (RNA targeting therapeutics: molecular mechanisms of antisense oligonucleotides as a therapeutic platform. Annu Rev Pharmacol Toxicol. 2010; 50:259-93); Wan and Seth, 2016 (The Medicinal Chemistry of Therapeutic Oligonucleotides. J Med Chem. 2016 Nov. 10; 59(21):9645-9667); Juliano, 2016 (The delivery of therapeutic oligonucleotides. Nucleic Acids Res. 2016 Aug. 19; 44(14):6518-48); Lundin et al., 2015 (Oligonucleotide Therapies: The Past and the Present. Hum Gene Ther. 2015 August; 26(8):475-85); and Prakash, 2011 (An overview of sugar-modified oligonucleotides for antisense therapeutics. Chem Biodivers. 2011 September; 8(9):1616-41). Indeed, for use in vivo, the oligonucleotide may be stabilized. A “stabilized” oligonucleotide refers to an oligonucleotide that is relatively resistant to in vivo degradation (e.g. via an exo- or endo-nuclease). Stabilization can be a function of length or secondary structure. In particular, oligonucleotide stabilization can be accomplished via phosphate backbone modifications, phosphodiester modifications, phosphorothioate (PS) backbone modifications, combinations of phosphodiester and phosphorothioate modifications, thiophosphoramidate modifications, 2′ modifications (2′-0-Me, 2′-O-(2-methoxyethyl) (MOE) modifications and 2′-fluoro modifications), methylphosphonate, methylphosphorothioate, phosphorodithioate, p-ethoxy, and combinations thereof.

In a particular embodiment, the antisense oligonucleotide is lipid-conjugated, known as LASO. In some embodiments, the antisense oligonucleotide of the present invention is modified by substitution at the 3′ or the 5′ end by a moiety comprising at least three saturated or unsaturated, particularly saturated, linear or branched, particularly linear, hydrocarbon chains comprising from 2 to 30 carbon atoms, particularly from 5 to 20 carbon atoms, more particularly from 10 to 18 carbon atoms as described in WO2014/195432.

In some embodiments, the antisense oligonucleotide of the present invention is modified by substitution at the 3′ or the 5′ end by a moiety comprising at least one ketal functional group, wherein the ketal carbon of said ketal functional group bears two saturated or unsaturated, particularly saturated, linear or branched, particularly linear, hydrocarbon chains comprising from 1 to 22 carbon atoms, particularly from 6 to 20 carbon atoms, in particular 10 to 19 carbon atoms, and even more particularly from 12 to 18 carbon atoms as described in WO2014/195430.

For example, the oligonucleotide may be employed as phosphorothioate derivatives (replacement of a non-bridging phosphoryl oxygen atom with a sulfur atom), which have increased resistance to nuclease digestion. 2′-methoxyethyl (MOE) modification (such as the modified backbone commercialized by IONIS Pharmaceuticals) is also effective. Additionally or alternatively, the oligonucleotide of the present invention may comprise completely, partially or in combination, modified nucleotides which are derivatives with substitutions at the 2′ position of the sugar, in particular with the following chemical modifications: O-methyl group (2′-O-Me) substitution, 2-methoxyethyl group (2′-O-MOE) substitution, fluoro group (2′-fluoro) substitution, chloro group (2′-C1) substitution, bromo group (2′-Br) substitution, cyanide group (2′—CN) substitution, trifluoromethyl group (2′-CF3) substitution, OCF3 group (2′-OCF3) substitution, OCN group (2′-OCN) substitution, O-alkyl group (2′-O-alkyl) substitution, S-alkyl group (2′-S-alkyl) substitution, N-alkyl group (2′-N-akyl) substitution, O-alkenyl group (2′-O-alkenyl) substitution, S-alkenyl group (2′-S-alkenyl) substitution, N-alkenyl group (2′-N-alkenyl) substitution, SOCH3 group (2′-SOCH3) substitution, SO2CH3 group (2′-SO2CH3) substitution, ONO2 group (2′-ONO2) substitution, NO2 group (2′-NO2) substitution, N3 group (2′-N3) substitution and/or NH2 group (2′—NH2) substitution. Additionally or alternatively, the oligonucleotide of the present invention may comprise completely or partially modified nucleotides wherein the ribose moiety is used to produce locked nucleic acid (LNA), in which a covalent bridge is formed between the 2′ oxygen and the 4′ carbon of the ribose, fixing it in the 3′-endo configuration. These molecules are extremely stable in biological medium, able to activate RNase H such as when LNA are located to extremities (Gapmer) and form tight hybrids with complementary RNA and DNA.

In some embodiments, the oligonucleotide used in the context of the invention comprises modified nucleotides selected from the group consisting of LNA, 2′-OMe analogs, 2′-O-Met, 2′-O-(2-methoxyethyl) (MOE) oligomers, 2′-phosphorothioate analogs, 2′-fluoro analogs, 2′-C1 analogs, 2′-Br analogs, 2′-CN analogs, 2′-CF3 analogs, 2′-OCF3 analogs, 2′-OCN analogs, 2′-O-alkyl analogs, 2′-S-alkyl analogs, 2′-N-alkyl analogs, 2′-O-alkenyl analogs, 2′-S-alkenyl analogs, 2′-N-alkenyl analogs, 2′-SOCH3 analogs, 2′-SO2CH3 analogs, 2′-ONO2 analogs, 2′-NO2 analogs, 2′-N3 analogs, 2′-NH2 analogs, tricyclo (tc)-DNAs, U7 short nuclear (sn) RNAs, tricyclo-DNA-oligoantisense molecules and combinations thereof (U.S. Provisional patent application Ser. No. 61/212,384 For: Tricyclo-DNA Antisense Oligonucleotides, Compositions and Methods for the Treatment of Disease, filed Apr. 10, 2009, the complete contents of which is hereby incorporated by reference).

In a particular embodiment, the oligonucleotide according to the invention is a LNA oligonucleotide. As used herein, the term “LNA” (Locked Nucleic Acid) (or “LNA oligonucleotide”) refers to an oligonucleotide containing one or more bicyclic, tricyclic or polycyclic nucleoside analogues also referred to as LNA nucleotides and LNA analogue nucleotides. LNA oligonucleotides, LNA nucleotides and LNA analogue nucleotides are generally described in International Publication No. WO 99/14226 and subsequent applications; International Publication Nos. WO 00/56746, WO 00/56748, WO 00/66604, WO 01/25248, WO 02/28875, WO 02/094250, WO 03/006475; U.S. Pat. Nos. 6,043,060, 6,268,490, 6,770,748, 6,639,051, and U.S. Publication Nos. 2002/0125241, 2003/0105309, 2003/0125241, 2002/0147332, 2004/0244840 and 2005/0203042, all of which are incorporated herein by reference. LNA oligonucleotides and LNA analogue oligonucleotides are commercially available from, for example, Proligo LLC, 6200 Lookout Road, Boulder, Colo. 80301 USA.

Other forms of oligonucleotides of the present invention are oligonucleotide sequences coupled to small nuclear RNA molecules such as U1 or U7 in combination with a viral transfer method based on, but not limited to, lentivirus or adeno-associated virus (Denti, M A, et al, 2008; Goyenvalle, A, et al, 2004).

Other forms of oligonucleotides of the present invention are peptide nucleic acids (PNA). In peptide nucleic acids, the deoxyribose backbone of oligonucleotides is replaced with a backbone more akin to a peptide than a sugar. Each subunit, or monomer, has a naturally occurring or non-naturally occurring base attached to this backbone. One such backbone is constructed of repeating units of N-(2-aminoethyl)glycine linked through amide bonds. Because of the radical deviation from the deoxyribose backbone, these compounds were named peptide nucleic acids (PNAs) (Dueholm et al., New J. Chem., 1997, 21, 19-31). PNA binds both DNA and RNA to form PNA/DNA or PNA/RNA duplexes. The resulting PNA/DNA or PNA/RNA duplexes are bound with greater affinity than corresponding DNA/DNA, DNA/RNA or RNA/RNA duplexes as determined by Tm's. This high thermal stability might be attributed to the lack of charge repulsion due to the neutral backbone in PNA. The neutral backbone of the PNA also results in the Tm's of PNA/DNA(RNA) duplex being practically independent of the salt concentration. Thus the PNA/DNA(RNA) duplex interaction offers a further advantage over DNA/DNA, DNA/RNA or RNA/RNA duplex interactions which are highly dependent on ionic strength. Homopyrimidine PNAs have been shown to bind complementary DNA or RNA in an anti-parallel orientation forming (PNA)2/DNA(RNA) triplexes of high thermal stability (see, e.g., Egholm, et al., Science, 1991, 254, 1497; Egholm, et al., J. Am. Chem. Soc., 1992, 114, 1895; Egholm, et al., J. Am. Chem. Soc., 1992, 114, 9677). In addition to increased affinity, PNA has also been shown to bind to DNA or RNA with increased specificity. When a PNA/DNA duplex mismatch is melted relative to the DNA/DNA duplex there is seen an 8 to 20° C. drop in the Tm. This magnitude of a drop in Tm is not seen with the corresponding DNA/DNA duplex with a mismatch present. The binding of a PNA strand to a DNA or RNA strand can occur in one of two orientations. The orientation is said to be anti-parallel when the DNA or RNA strand in a 5′ to 3′ orientation binds to the complementary PNA strand such that the carboxyl end of the PNA is directed towards the 5′ end of the DNA or RNA and amino end of the PNA is directed towards the 3′ end of the DNA or RNA. In the parallel orientation the carboxyl end and amino end of the PNA are just the reverse with respect to the 5′-3′ direction of the DNA or RNA. A further advantage of PNA compared to oligonucleotides is that their polyamide backbones (having appropriate nucleobases or other side chain groups attached thereto) is not recognized by either nucleases or proteases and are not cleaved. As a result, PNAs are resistant to degradation by enzymes unlike nucleic acids and peptides. WO92/20702 describes a peptide nucleic acid (PNA) compounds which bind complementary DNA and RNA more tightly than the corresponding DNA. PNA have shown strong binding affinity and specificity to complementary DNA (Egholm, M., et al., Chem. Soc., Chem. Commun., 1993, 800; Egholm, M., et.al., Nature, 1993, 365, 566; and Nielsen, P., et.al. Nucl. Acids Res., 1993, 21, 197). Furthermore, PNA's show nuclease resistance and stability in cell-extracts (Demidov, V. V., et al., Biochem. Pharmacol., 1994, 48, 1309-1313). Modifications of PNA include extended backbones (Hyrup, B., et.al. Chem. Soc., Chem. Commun., 1993, 518), extended linkers between the backbone and the nucleobase, reversal of the amida bond (Lagriffoul, P. H., et.al., Biomed. Chem. Lett., 1994, 4, 1081), and the use of a chiral backbone based on alanine (Dueholm, K. L, et.al., BioMed. Chem. Lett., 1994, 4, 1077). Peptide Nucleic Acids are described in U.S. Pat. Nos. 5,539,082 and 5,539,083. Peptide Nucleic Acids are further described in U.S. patent application Ser. No. 08/686,113.

Typically, the oligonucleotides of the present invention are obtained by conventional methods well known to those skilled in the art. For example, the oligonucleotide of the invention can be synthesized de novo using any of a number of procedures well known in the art. For example, the b-cyanoethyl phosphoramidite method (Beaucage et al., 1981); nucleoside H-phosphonate method (Garegg et al., 1986; Froehler et al., 1986, Garegg et al., 1986, Gaffney et al., 1988). These chemistries can be performed by a variety of automated nucleic acid synthesizers available in the market. These nucleic acids may be referred to as synthetic nucleic acids. Alternatively, oligonucleotide can be produced on a large scale in plasmids (see Sambrook, et al., 1989). Oligonucleotide can be prepared from existing nucleic acid sequences using known techniques, such as those employing restriction enzymes, exonucleases or endonucleases. Oligonucleotide prepared in this manner may be referred to as isolated nucleic acids.

The one skilled in the art can easily provide some approaches and modifications for enhancing the delivery and the efficacy of oligonucleotides such as chemical modification of the oligonucleotides, lipid- and polymer-based nanoparticles or nanocarriers, ligand-oligonucleotide conjugates by linking oligonucleotides to targeting agents such as carbohydrates, peptides, antibodies, aptamers, lipids or small molecules and small molecules that improve oligonucleotide delivery such as described in Juliano R L. The delivery of therapeutic oligonucleotides. Nucleic Acids Res. 2016 Aug. 19; 44(14):6518-48. Lipophilic conjugates and lipid conjugates include fatty acid-oligonucleotide conjugates; sterol-oligonucleotide conjugates and vitamin-oligonucleotide conjugates.

In a particular embodiment, the oligonucleotide of the present invention is conjugated to a second molecule. Typically said second molecule is selected from the group consisting of aptamers, antibodies or polypeptides. For example, the oligonucleotide of the present invention may be conjugated to a cell penetrating peptide. Cell penetrating peptides are well known in the art and include for example the TAT peptide (Bechara C, Sagan S. Cell-penetrating peptides: 20 years later, where do we stand? FEBS Lett. 2013 Jun. 19; 587(12):1693-702).

In some embodiments, the oligonucleotide of the present invention is associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the oligonucleotide, or improve the oligonucleotide's pharmacokinetic or therapeutic properties. For example, the oligonucleotide of the present invention may also be administered encapsulated in liposomes, pharmaceutical compositions wherein the active ingredient is contained either dispersed or variously present in corpuscles consisting of aqueous concentric layers adherent to lipidic layers. The oligonucleotide, depending upon solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such as diacetylphosphate, stearylamine, or phosphatidic acid, or other materials of a hydrophobic nature. The diameters of the liposomes generally range from about 15 nm to about 5 microns. The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids, which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a liposome delivery vehicle originally designed as a research tool, such as Lipofectin, can deliver intact nucleic acid molecules to cells. Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

In some embodiments, the oligonucleotide of the present invention is complexed with a complexing agent to increase cellular uptake of oligonucleotides. An example of a complexing agent includes cationic lipids. Cationic lipids can be used to deliver oligonucleotides to cells. The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Particularly, straight chain or branched alkyl or alkene groups have six or more carbon atoms. Alicyclic groups include cholesterol and other steroid groups. Cationic lipids can be prepared with a variety of counterions (anions) including, e.g., Cl—, Br—, I—, F—, acetate, trifluoroacetate, sulfate, nitrite, and nitrate. Examples of cationic lipids include: polyethylenimine, polyamidoamine (PAMAM) starburst dendrimers, Lipofectin (a combination of DOTMA and DOPE), Lipofectase, Lipofectamine, DOPE, Cytofectin (Gilead Sciences, Foster City, Calif.), and Eufectins (JBL, San Luis Obispo, Calif.). Cationic liposomes may comprise the following: N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3p-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethy-1-hydroxyethyl ammonium bromide; and dimethyldioctadecylammonium bromide (DDAB). The cationic lipid N-(1-(2,3-dioleyloxy)propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108). Oligonucleotides can also be complexed with, e.g., poly(L-lysine) or avidin and lipids may, or may not, be included in this mixture (e.g., steryl-poly(L-lysine). Cationic lipids have been used in the art to deliver oligonucleotides to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligonucleotides can be used in connection with the claimed methods. In addition to those listed supra, other lipid compositions are also known in the art and include, e.g., those taught in U.S. Pat. Nos. 4,235,871; 4,501,728; 4,837,028; 4,737,323.

In a particular embodiment, the antisense oligonucleotide according to the invention comprises a sequence consisting of any sequences of SEQ ID NO:1 to SEQ ID NO:93.

In a particular embodiment, the antisense oligonucleotide according to the invention consists of a sequence consisting of any sequences of SEQ ID NO:1 to SEQ ID NO:93.

In a particular embodiment, the inhibitor and/or the antisense oligonucleotide according to the invention is capable of reducing the amount of DDX5 in cancer cells.

In a particular embodiment, the invention relates to an ASO having at least 70% of identity with an ASO of SEQ ID NO: 1 to SEQ ID NO:93. In a particular embodiment the percentage of identity can be 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99%. Nucleic acid sequence identity is particularly determined using a suitable sequence alignment algorithm and default parameters, such as BLAST N (Karlin and Altschul, Proc. Natl Acad. Sci. USA 87(6):2264-2268 (1990)).

### Vector of the Invention

In a second aspect, the present invention relates to a vector for delivery of a heterologous nucleic acid, wherein the nucleic acid encodes an inhibitor according to the invention.

In a particular embodiment, the invention relates to a vector for delivery of a heterologous nucleic acid, wherein the nucleic acid encodes for an inhibitor according to the invention that specifically binds to DDX5 mRNA and inhibits expression of DDX5 in a cell.

In a particular embodiment, the vector according to invention, wherein the inhibitor is a siRNA or an antisense oligonucleotide as described above.

In a further embodiment, the acid nucleic acid (e.g. antisense nucleic acid) of the invention may be delivered in vivo alone (naked ASO/LASO) or in association with a vector.

In its broadest sense, a “vector” is any vehicle capable of facilitating the transfer of the oligonucleotide of the invention to the cells. Particularly, the vector transports the nucleic acid to cells with reduced degradation relative to the extent of degradation that would result in the absence of the vector. In general, the vectors useful in the invention include, but are not limited to, naked plasmids, non-viral delivery systems (cationic transfection agents, liposomes, lipid nanoparticles, and the like), phagemids, viruses, other vehicles derived from viral or bacterial sources that have been manipulated by the insertion or incorporation of the oligonucleotide sequences. Viral vectors include, but are not limited to nucleic acid sequences from the following viruses: RNA viruses such as a retrovirus (as for example moloney murine leukemia virus and lentiviral derived vectors), harvey murine sarcoma virus, murine mammary tumor virus, and rous sarcoma virus; adenovirus, adeno-associated virus (AAV); SV40-type viruses; polyoma viruses; Epstein-Barr viruses; papilloma viruses; herpes virus; vaccinia virus; polio virus. One can readily employ other vectors not named but known to the art.

Accordingly, an object of the invention relates to a vector comprising an oligonucleotide sequence that encodes a portion or fragment of DDX5, or variants thereof.

In another embodiment, the vector of the invention comprises any variant of the oligonucleotide sequence that encodes a portion or fragment of DDX5.

In another embodiment, the vector of the invention comprises any variant of the oligonucleotide sequence that encodes any variant of DDX5.

In another embodiment, the invention relates to a vector comprising an antisense oligonucleotide sequence that encodes a portion or fragment of DDX5, or variants thereof.

In another embodiment, the invention relates to a vector comprising a shRNA sequence that encodes a portion or fragment of the DDX5, or variants thereof.

In another embodiment, the invention relates to a vector comprising a miRNA sequence that encodes a portion or fragment of DDX5, or variants thereof.

In another embodiment, the vector according to the invention comprises an antisense oligonucleotide which targets the region comprising or consisting of the nucleic acids 276-515, 1056-1155, 1396-1795 or 1856-1955 of SEQ ID NO: 94.

In another embodiment, the invention relates to a vector comprising or consisting of any sequences from SEQ ID NO:1 to SEQ ID NO:93.

In another embodiment, the invention relates to a vector comprising an oligonucleotide sequence that encodes a portion or fragment of DDX5, or variants thereof and a CAG promoter.

In another embodiment, the invention relates to a vector comprising a miRNA sequence that encodes a portion or fragment of DDX5, or variants thereof and a CAG promoter or a PolII promoter.

In another embodiment, the invention relates to a vector comprising a shRNA sequence that encodes a portion or fragment of DDX5, or variants thereof and a U6 promoter.

The variants include, for instance, naturally-occurring variants due to allelic variations between individuals (e.g., polymorphisms), alternative splicing forms, etc. The term variant also includes genes sequences of the invention from other sources or organisms. Variants are preferably substantially homologous to sequences according to the invention, i.e., exhibit a nucleotide sequence identity of typically at least about 75%, preferably at least about 85%, more preferably at least about 90%, more preferably at least about 95% with sequences of the invention. Variants of the genes of the invention also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

In a particular embodiment, the vector use according to the invention is a non-viral vector or a viral vector.

In a particular embodiment, the non-viral vector is a plasmid comprising a nucleic acid sequence that encodes DDX5.

In another particular embodiment, the vector may a viral vector.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

As used herein, the term “transgene” refers to the antisense oligonucleotide of the invention.

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e. g. episomes), or integration of transferred genetic material into the genomic DNA of host cells.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478.

In a particular embodiment, the viral vector may be an adenoviral, a retroviral, a lentiviral, a herpesvirus or an adeno-associated virus (AAV) vectors.

In a particular embodiment, adeno-associated viral (AAV) vectors are employed.

In another embodiment, the invention relates to an adeno-associated virus (AAV) vector comprising an oligonucleotide sequence that targets a portion or fragment DDX5, or variants thereof.

In one embodiment, the AAV vector is AAV1, AAV2, AAV3, AAV4, AAS, AAV6, AAV7, AAV8, AAV9, AAV10, AAVrh.10 or any other serotypes of AAV that can infect human, rodents, monkeys or other species.

By an “AAV vector” is meant a vector derived from an adeno-associated virus serotype, including without limitation, AAV-1, AAV-2, AAV-3, AAV-4, AAV-5, AAV6, AAV7, AAV8, AAV9, AAV10, AAVrh.10, etc. AAV vectors can have one or more of the AAV wild-type genes deleted in whole or part, e.g. the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. ITRs do not need to be the wild-type polynucleotide sequences, and may be altered, e.g, by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest (i.e. the nucleic acid sequences of the invention) and a transcriptional termination region.

In certain embodiments the viral vectors utilized in the compositions and methods of the invention are recombinant adeno-associated virus (rAAV). The rAAV may be of any serotype, modification, or derivative, known in the art, or any combination thereof (e.g., a population of rAAV that comprises two or more serotypes, e.g., comprising two or more of rAAV2, rAAV8, and rAAV9) known in the art. In some embodiments, the rAAV are rAAV1, rAAV2, rAAV3, rAAV4, rAAV5, rAAV6, rAAV7, rAAV8, rAAV9, rAAV10, rAAV-11, rAAV-12, rAAV-13, rAAV-14, rAAV-15, rAAV-16, rAAV.rh8, rAAV.rh10, rAAV.rh20, rAAV.rh39, rAAV.Rh74, rAAV.RHM4-1, AAV.hu37, rAAV.Anc80, rAAV.Anc80L65, rAAV.7m8, rAAV.PHP.B, rAAV2.5, rAAV2tYF, rAAV3B, rAAV.LK03, rAAV.HSC1, rAAV.HSC2, rAAV.HSC3, rAAV.HSC4, rAAV.HSC5, rAAV.HSC6, rAAV.HSC7, rAAV.HSC8, rAAV.HSC9, rAAV.HSC10, rAAV.HSC11, rAAV.HSC12, rAAV.HSC13, rAAV.HSC14, rAAV.HSC15, or rAAV.HSC16, or other rAAV, or combinations of two or more thereof.

In some embodiments, the rAAV used in the compositions and methods of the invention comprise a capsid protein from an AAV capsid serotype selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, the rAAV comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., vp1, vp2 and/or vp3 sequence of an AAV capsid serotype selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, the AAV that is used in the methods described herein is Anc80 or Anc80L65, as described in Zinn et al., 2015: 1056-1068, which is incorporated by reference in its entirety. In certain embodiments, the AAV that is used in the methods described herein comprises one of the following amino acid insertions: LGETTRP (SEQ ID NO: 14) or LALGETTRP (SEQ ID NO: 15), as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV-PHP.B. In certain embodiments, the AAV that is used in the methods described herein is any AAV disclosed in U.S. Pat. No. 9,840,719 and WO 2015/013313, such as AAV.Rh74 and RHM4-1, each of which is incorporated herein by reference in its entirety. In certain embodiments, the AAV that is used in the methods described herein is any AAV disclosed in WO 2014/172669, such as AAV rh.74, which is incorporated herein by reference in its entirety. In certain embodiments, the AAV that is used in the methods described herein is AAV2/5, as described in Georgiadis et al., 2016, Gene Therapy 23: 857-862 and Georgiadis et al., 2018, Gene Therapy 25: 450, each of which is incorporated by reference in its entirety. In certain embodiments, the AAV that is used in the methods described herein is any AAV disclosed in WO 2017/070491, such as AAV2tYF, which is incorporated herein by reference in its entirety. In certain embodiments, the AAV that is used in the methods described herein is AAVLK03 or AAV3B, as described in Puzzo et al., 2017, Sci. Transl. Med. 29(9): 418, which is incorporated by reference in its entirety. In certain embodiments, the AAV that is used in the methods described herein is any AAV disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In certain embodiments, the AAV that is used in the methods described herein is an AAV disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, the rAAV have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the vp1, vp2 and/or vp3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, the rAAV have a capsid protein disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689, (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10), the contents of each of which is herein incorporated by reference in its entirety. In some embodiments, the rAAV have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the vp1, vp2 and/or vp3 sequence of an AAV capsid disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689 (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

In additional embodiments, the rAAV comprise a pseudotyped rAAV. In some embodiments, the pseudotyped rAAV are rAAV2/8 or rAAV2/9 pseudotyped rAAV. Methods for producing and using pseudotyped rAAV are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, the rAAV comprise a capsid containing a capsid protein chimeric of two or more AAV capsid serotypes. In some embodiments, the capsid protein is a chimeric of 2 or more AAV capsid proteins from AAV serotypes selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In certain embodiments, the recombinant AAV vector used for delivering the transgene have a tropism for cells in the DRG. Such vectors can include non-replicating “rAAV”, particularly those bearing an AAV8 or AAVrh10 capsid are preferred. In certain embodiments, the viral vectors provided herein are AAV9 or AAVrh10 based viral vectors. In certain embodiments, the AAV8 or AAVrh10 based viral vectors provided herein retain tropism for DRG. AAV variant capsids can be used, including but not limited to those described by Wilson in U.S. Pat. No. 7,906,111 which is incorporated by reference herein in its entirety, with AAV/hu.31 and AAV/hu.32 being particularly preferred; as well as AAV variant capsids described by Chatterjee in U.S. Pat. Nos. 8,628,966, 8,927,514 and Smith et al., 2014, Mol Ther 22: 1625-1634, each of which is incorporated by reference herein in its entirety. In some embodiment, the present invention relates to a recombinant adeno-associated virus (rAAV) comprising (i) an expression cassette containing a transgene under the control of regulatory elements and flanked by ITRs, and (ii) an AAV capsid, wherein the transgene encodes an inhibitory RNA that specifically binds DDX5 mRNA and inhibits expression of DDX5 in a cell.

Provided in particular embodiments are AAV vectors comprising an artificial genome comprising (i) an expression cassette containing the transgene under the control of regulatory elements and flanked by ITRs; and (ii) a viral capsid that has the amino acid sequence of the AAV capsid protein or is at least 95%, 96%, 97%, 98%, 99% or 99.9% identical to the amino acid sequence of the AAV capsid protein while retaining the biological function of the AAV capsid.

Provided in particular embodiments are AAVrh10 vectors comprising an artificial genome comprising (i) an expression cassette containing the transgene under the control of regulatory elements and flanked by ITRs; and (ii) a viral capsid that has the amino acid sequence of the AAVrh10 capsid protein or is at least 95%, 96%, 97%, 98%, 99% or 99.9% identical to the amino acid sequence of the AAVrh10 capsid protein while retaining the biological function of the AAVrh10capsid. In certain embodiments, the encoded AAVrh10 capsid has the sequence of SEQ ID NO: 81 set forth in U.S. Pat. No. 9,790,427 which is incorporated by reference herein in its entirety, with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAVrh10 capsid.

The control elements are selected to be functional in a mammalian cell. The resulting construct which contains the operatively linked components is flanked by (5′ and 3′) functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a polynucleotide sequence interposed between two flanking ITRs into a mammalian cell genome. The polynucleotide sequences of AAV ITR regions are known. As used herein, an “AAV ITR” does not necessarily comprise the wild-type polynucleotide sequence, but may be altered, e. g., by the insertion, deletion or substitution of nucleotides. Additionally, the AAV ITR may be derived from any of several AAV serotypes, including without limitation, AAV-1, AAV-2, AAV-3, AAV-4, AAV-5, AAV6, AAV7, AAV8, AAV9, AAV10, AAVrh.10, etc. Furthermore, 5′ and 3′ ITRs which flank a selected polynucleotide sequence in an AAV vector need not necessarily be identical or derived from the same AAV serotype or isolate, so long as they function as intended, i. e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV 5, AAV6, AAV7, AAV8, AAV9, AAV10, AAVrh.10, etc. Furthermore, 5′ and 3′ ITRs which flank a selected polynucleotide sequence in an AAV expression vector need not necessarily be identical or derived from the same AAV serotype or isolate, so long as they function as intended, i. e., to allow for excision and rescue of the sequence of interest from a host cell genome or vector, and to allow integration of the DNA molecule into the recipient cell genome when AAV Rep gene products are present in the cell.

Particular embodiments are vectors derived from AAV serotypes having tropism for and high transduction efficiencies in cells of the mammalian DRG. A review and comparison of transduction efficiencies of different serotypes is provided in this patent application. In certain examples, AAV2, AAV5, AAV8, AAV9 and rh.10 based vectors direct long-term expression of transgenes in DRG.

The selected polynucleotide sequence is operably linked to control elements that direct the transcription or expression thereof in the subject in vivo. Such control elements can comprise control sequences normally associated with the selected gene.

Typically the vector of the present invention comprises an expression cassette. The term “expression cassette” refers to a nucleic acid construct comprising nucleic acid elements sufficient for the expression of the nucleic acid molecule of the present invention. Typically the nucleic acid molecule encodes a heterologous gene and may also include suitable regulatory elements. The heterologous gene refers to a transgene that encodes an RNA of interest.

One or more expression cassettes may be employed. Each expression cassette may comprise at least a promoter sequence operably linked to a sequence encoding the RNA of interest. Each expression cassette may consist of additional regulatory elements, spacers, introns, UTRs, polyadenylation site, and the like. In some embodiments, the expression cassette is polycistronic with respect to the transgenes encoding e.g. two or more miRNAs. In other embodiments the expression cassette comprises a promoter, a nucleic acid encoding one or more RNA molecules of interest, and a polyA. In further embodiments, the expression cassette comprises 5′-promoter sequence, a sequence encoding a first RNA of interest, a sequence encoding a second RNA of interest, and a polyadenylation sequence-3′.

In some embodiments, an expression cassette may comprise additional elements, for example, an intron, an enhancer, a polyadenylation site, a woodchuck posttranscriptional response element (WPRE), and/or other elements known to affect expression levels of the encoding sequence. Typically, an expression cassette comprises the nucleic acid molecule of the present invention operatively linked to a promoter sequence.

The term “operatively linked” refers to the association of two or more nucleic acid fragments on a single nucleic acid fragment so that the function of one is affected by the other.

For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation.

As used herein, the term “promoter” sequence refers to a polynucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In some embodiments, the promoter is a heterologous promoter. The term “heterologous promoter”, as used herein, refers to a promoter that is not found to be operatively linked to a given encoding sequence in nature.

Useful heterologous control sequences generally include those derived from sequences encoding mammalian or viral genes. Examples include, but are not limited to, the phophoglycerate kinase (PKG) promoter, CAG (composite of the (CMV) cytomegalovirus enhancer the chicken beta actin promoter (CBA) and the rabbit beta globin intron), U6 promoter, neuronal promoters (Human synapsin 1 (hSyn) promoter, NeuN promoters, CamKII promoter, promoter of Dopamine-1 receptor and Dopamine-2 receptor), the SV40 early promoter, mouse mammary tumor virus LTR promoter; adenovirus major late promoter (Ad MLP); a herpes simplex virus (HSV) promoter, a CMV promoter such as the CMV immediate early promoter region (CMV-IE), rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

For purposes of the present invention, both heterologous promoters and other control elements, such as DRG-specific and inducible promoters, enhancers and the like, will be of particular use.

An “enhancer” is a polynucleotide sequence that can stimulate promoter activity and may be an innate element of the promoter or a heterologous element inserted to enhance the level or tissue-specificity of a promoter. In some embodiments, the promoter is derived in its entirety from a native gene. In some embodiments, the promoter is composed of different elements derived from different naturally occurring promoters. In some embodiments, the promoter comprises a synthetic polynucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g. tetracycline-responsive promoters) are well known to those of skill in the art.

In mammalian systems, three kinds of promoters exist and are candidates for construction of the expression vectors: Pol I promoters control transcription of large ribosomal RNAs; Pol II promoters control the transcription of mRNAs (that are translated into protein) and small nuclear RNAs (snRNAs); and Pol III promoters uniquely transcribe small non-coding RNAs. Each has advantages and constraints to consider when designing the construct for expression of the RNAs in vivo. For example, Pol III promoters are useful for synthesizing small interfering RNAs (shRNAs) from DNA templates in vivo. For greater control over tissue specific expression, Pol II promoters are preferred but can only be used for transcription of miRNAs. When a Pol II promoter is used, however, it may be preferred to omit translation initiation signals so that the RNAs function as antisense, siRNA, shRNA or miRNAs and are not translated into peptides in vivo.

The AAV expression vector which harbors the DNA molecule of interest flanked by AAV ITRs, can be constructed by directly inserting the selected sequence (s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publications Nos. WO 92/01070 (published 23 Jan. 1992) and WO 93/03769 (published 4 Mar. 1993). Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian DRG cells can be used, and in certain embodiments codon optimization of the transgene is performed by well-known methods. The complete chimeric sequence is assembled from overlapping oligonucleotides prepared by standard methods. In order to produce AAV virions, an AAV expression vector is introduced into a suitable host cell using known techniques, such as by transfection. A number of transfection techniques are generally known in the art. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

For instance, a particular viral vector comprises, in addition to a nucleic acid sequence of the invention, the backbone of AAV vector plasmid with ITR derived from AAV-2, the promoter, such as the mouse PGK (phosphoglycerate kinase) gene or the cytomegalovirus/β-actin hybrid promoter (CAG) consisting of the enhancer from the CMV immediate gene, the promoter, splice donor and intron from the chicken β-actin gene, the splice acceptor from rabbit β-globin, or any neuronal promoter such as the promoter of Dopamine-1 receptor or Dopamine-2 receptor, or the synapsin promoter, with or without the wild-type or mutant form of woodchuck hepatitis virus post-transcriptional regulatory element (WPRE), and a rabbit beta-globin polyA sequence. The viral vector may comprise in addition, a nucleic acid sequence encoding an antibiotic resistance gene such as the genes of resistance ampicillin (AmpR), kanamycin, hygromycin B, geneticin, blasticidin S or puromycin.

In one embodiment, retroviral vectors are employed.

Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types.

In another embodiment, lentiviral vectors are employed.

In a particular embodiment, the invention relates to a lentivirus vector comprising an oligonucleotide sequence that targets a portion or fragment of DDX5, or variants thereof.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the nucleic acid molecule or the vector of the present invention include “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

Method for Treating Cancer

In a third aspect, the invention relates to the inhibitor and/or the antisense oligonucleotide as described above for use in the treatment of cancer in a subject in need thereof.

In a particular embodiment, the invention relates to a method for treating cancer in a subject in need thereof comprising a step of administering said subject with a therapeutically effective amount of an inhibitor and/or the antisense oligonucleotide as described above.

According to the invention, the cancer may be selected in the group consisting of adrenal cortical cancer, anal cancer, bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

In a particular embodiment the cancer is a prostate cancer, a resistant (to chemotherapy or radiotherapy) prostate cancer or a Castration Resistant Prostate Cancer (CRPC).

In a particular embodiment, the method according to the invention wherein said antisense oligonucleotide is administered alone (naked) or in a vector as described above.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “subject” denotes a mammal, such as a rodent, a feline, a canine, and a primate. Particularly, the subject according to the invention is a human, a mouse or a rat. As used herein, the term “subject” encompasses “patient”.

In a particular embodiment, the subject suffers or is susceptible to suffer from a cancer and particularly to a prostate cancer.

As used herein the terms “administering” or “administration” refer to the act of injecting or otherwise physically delivering a substance as it exists outside the body (e.g., an inhibitor of DDX5 such as an ASO of the invention) into the subject, such as by, intravenous, intramuscular, enteral, subcutaneous, parenteral, systemic, local, spinal, nasal, topical or epidermal administration (e.g., by injection or infusion). When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof. In a particular embodiment, the administration is performed by a patches, a paste, an ointment, a suspension, a solution or a cream, a gel or a spray. In a particular embodiment, the administration is performed by a cream.

In a particular embodiment, the administration of the inhibitor and/or antisense oligonucleotide is performed by an intrathecal, subcutaneous, topical or intravenous administration.

In a further embodiment, i) an antisense oligonucleotide according to the invention and and a ii) classical treatment for simultaneous, separate or sequential use in the treatment of cancer as a combined preparation.

As used herein, the term “classical treatment” refers to any compound, natural or synthetic. In a particular embodiment, the classical treatment is selected from the group consisting of but not limited to: aspirin, paracetamol, Nonsteroidal anti-inflammatory drugs (NSAIDs); codeine, cryotherapy, virtual therapy, cannabis, morphine and its derivatives, opium and its derivatives.

A “therapeutically effective amount” is intended for a minimal amount of active agent (e.g. ASO according to the invention) which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Composition

In a fourth aspect, the invention relates to a pharmaceutical composition which comprises the inhibitor and/or the antisense oligonucleotide according to the invention.

In a particular embodiment, the invention relates to the pharmaceutical composition according to the invention for use in the treatment of cancer.

The inhibitor and/or the antisense oligonucleotide as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for per os (oral), sublingual, subcutaneous, intramuscular, intravenous, transdermal, local or rectal administration, the active principle, alone or in combination with another active principle, can be administered in a unit administration form, as a mixture with conventional pharmaceutical supports, to subjects, such as animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

In a particular embodiment, the pharmaceutical composition according to the invention is administered by an intrathecal, subcutaneous, topical or intravenous administration.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation may be vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In a particular embodiment, the present invention provides a topical formulation comprising antisense oligonucleotides. For example, and not by way of limitation, the present invention provides a topical formulation comprising antisense oligonucleotides. Dosage forms for the topical or transdermal administration of the inhibitors of the present invention include, but are not limited to, powders, sprays, ointments, pastes, creams, lotions, gels, solutions, patches and inhalants. In certain non-limiting embodiments, a topical formulation comprises antisense oligonucleotides comprised in micelles, liposomes, or non-lipid based microspheres. In certain non-limiting embodiments, such a topical formulation may comprise a permeability enhancing agent such as but not limited to dimethyl sulfoxide, hydrocarbons (for example, alkanes and alkenes), alcohols (for example, glycols and glycerols), acids (for example, fatty acids), amines, amides, esters (for example, isopropyl myristate), surfactants (for example, anionic, cationic, or non-ionic surfactants), terpenes, and lipids (for example, phospholipids).

In a particular embodiment, the formulation is a patches, a paste, an ointment, a suspension, a solution or a cream, a gel or a spray. In a particular embodiment, the formulation is a cream.

Pharmaceutical compositions of the present invention may comprise a further therapeutic active agent. The present invention also relates to a kit comprising an agonist, antagonist or inhibitor of the expression according to the invention and a further therapeutic active agent.

For example, anti-cancer agents may be added to the pharmaceutical composition as described below.

Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiri de, tetrahydrocannabinols, thiefhylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

The invention will be further illustrated by the following figures and examples. However, these examples and figures should not be interpreted in any way as limiting the scope of the present invention.

### FIGURES

FIG. 1. Developing Anti Sense Oligonucleotide (ASO) inhibiting DDX5 protein levels in dose-dependency manner

A, The first screening to define the ASO inhibiting DDX5 protein expression. The hASO #51 was found to decrease DDX5 protein level in dose-dependency manner. B, The hASO #51 inhibited cell proliferation of the DU 145 cells. The MTT test was done with biological triplication. ** The data was analyzed using independent sample t-test.t value=5.513, p=0.00528. C and D, The second screening testing 8 different bi-specific ASO (hmASOs) along with the hASO #51 as a positive control. ASO #3, #55, #71 displayed to deplete DDX5 protein levels significantly. Further experiment showing that hmASO #3 is able to decrease DDX5 expression.

FIG. 2: Testing different batches of ASO3 and ASO51 to inhibit DDX5 protein levels.

ASO3 old/new/vivo are the same ASO3 (SEQ ID NO:3) and from just different synthesis batches. ASO51 old/new are the same ASO51 (SEQ ID NO:51) and from just different synthesis batches. The protocol for ASO transfection is the same as reported in Material & Methods.

FIG. 3: WB testing various ASOs that are nearby the ASO3.

ASO3 (SEQ ID NO:3) and nearby ASO3 are tested at a concentration at 120 nM. ASO3-1, ASO+1 and ASO3+3 also inhibit DDX5 protein level beside the ASO3.

### EXAMPLE

Material & Methods

Cell Lines and Cell Culture Condition

We had different human prostate cell lines in our study: the normal phenotype cell PNT1A (ECACC, European Collection of Cell Cultures, England), castration sensitive (CS) LNCaP (ATCC, American Type Culture Collection (Rockville, Md., USA)), and castration-resistant (CR) DU145 and PC3 cell lines (ATCC). We also used two LNCaP-derived cell lines, LNCaP Mock and LNCaP Hsp27 which were established as described (Baylot et al. 2012). The cells were maintained in either the RPMI-1640 medium (Roswell Park Memorial Institute) (PNT1A, LNCaP, LNCaP Mock, LNCaP Hsp27) or DMEM medium (Dulbecco's Modified Eagle's Medium) (PC3, DU145), supplemented 10% fetal bovine serum (FBS) at 37° C. in 5% CO2.

Human Prostate Cancer TMA Construction and Immunohistochemistry (IHC)

A total of 352 specimens [33 benign prostate tumours, 94 primary tumours without lymph node metastasis, 61 primary tumours with lymph node metastasis, 78 neo-adjuvant-treated primary tumours, 86 castration-resistant prostate cancers (CRPC)] were collected from the Vancouver Prostate Centre Tissue Bank. The TMA construction has previously been described (Thomas et al. 2011). IHC staining with mouse anti-DDX5 monoclonal antibody (sc-365164, Santa Cruz Biotechnology, CA, USA) was performed using a Ventana autostainer (model Discover XT; Ventana Medical System, Tucson, Ariz.) with an enzyme-labelled biotin-streptavidin system and a solvent-resistant DAB Map kit (Ventana). We quantified the staining of DDX5 on each cores with the Quick Score (QS) which combined the intensity of the staining and the percentage of stained cells. The level of DDX5 immunostaining was scored on a scale from 0 to 3 by pathologist as described (Choi et al. 2016). Basically, 0 was undetectable stain, 1 means a faint stain, 2 means a stain with obvious intensity occurring in a minority of cells, and 3 represents for a stain with convincing intensity in almost of cells.

Development of DDX5-Targeting ASOs

Developing DDX5-tageting ASOs has been performing in the “Optoligo” national platform which is sponsored by Inserm Transfert. The sequence of ASOs was designed by using an informatics R script which created by Pascal FINETTI in our laboratory. Firstly, the coding part of DDX5 mRNA sequence (CCDS11659.1) was selected and segmented into consecutive sequences of 20 bases. The resulting sequences were converted into their complementary acid nucleotide sequences, which subsequently were inverted to obtain the 5′ to 3′ format and become potential ASO sequences. In a second step, the ASO sequences were individually evaluated on the percentage of GC and the specificity to the input transcripts. The specificity evaluation was performed by using NCBI's ‘Basic Local Alignment Search Tool’ (BLAST), with the use of the ‘blastn’ algorithm and the NCBI reference transcript database ‘refseq_rna’ as parameters. The next selection was done manually by excluding ASOs showing sequence similarities with other genes. Finally, the selected ASOs were synthesized by Pr. Philippe BARTHELEMY (ARNA Laboratory, Inserm U1212, CNRS UMR5320, University of Bordeaux). The biological activity of the different ASOs was then tested on PC tumor cell line, the inhibition of DDX5 protein was evaluated by Western blot (WB) and then quantified with Image J (NIH) software.

Treatment Cells with ASOs

Seeding of the cells was carried out 1 day before treatment at a density of 2300 cells per 1 cm². The cells were treated with indicated ASO concentrations after incubating with 3 mg/ml of Oligofactamine (Invitrogen) in serum free OPTI-MEM medium (Invitrogen) for 20 minutes. After 4-5 hours, the transfection mixtures were removed and replaced by the complete medium. One day later, the second treatment was performed identically. The transfected cells were collected after 3 days of the latest ASO transfection. We used scrambled ASO as control.

Immunoprecipitation Coupled Mass Spectrometry (IP/MS)

An amount of 2 mg protein extracts from 4 cell lines (PNT1a, LNCaP, DU 145, and PC 3) was diluted with the lysis buffer to obtain the final concentration of 4 mg protein per 1 ml. Subsequently, the protein extracts were pre cleaned with 40 μl of the protein A Sepharose (nProtein A Sepharose® 4 Fast Flow, REF.17-5280-01, GE Healthcare, MERCK). Subsequently, the lysate was incubated with 5 μg of Ab against DDX5 (mouse monoclonal Ab, sc-365164, Santa Cruz Biotechnology) overnight at 4° C. The immunoprecipitated complexes were then captured by incubation with 40 μl of protein A Sepharose bead for 1 hr, 4° C., which was followed by 3 times of washing using the lysis buffer. Ultimately, the resulting beads were suspended with 20 μl Laemmli sample buffer 4×, heated at 95° C. for 5 minutes. To evaluate the efficiency of the IP, 10% of the samples were run on the SDS-PAGE gel for silver staining analysis as described (Chevallet et al. 2006). The proteins in immunoprecipitated complexes were determined by LC-MS/MS using LTQ Orbitrap (Thermoscientific). The IP experiments were done with triplications and 3 technical replications.

Western Blot Analysis (WB)

The total protein extracts were obtained by resuspension the cell pellet with the lysis buffer (1% v/v Triton X-100, 50 mM HEPES, 150 mM NaCl, 25 mM NaF, 1 mM EDTA, 1 mM EGTA, 10 μM ZnCl2, 1 mM sodium orthovanadate) containing 4% v/v protease inhibitor cocktail (Roche) and incubated for 20 minutes on ice. After centrifugation at 13 000 rpm, 4° C., 30 minutes, the clear lysate was collected and quantified by using BCA protein assay kit (Pierce). The protein (20 μg per lane) was pre mixed with the Laemmli sample buffer 4× and boiled at 95° C. for 5 minutes before running on the SDS-polyacrylamide gels (10%). The migrated proteins were then transferred into the polyvinylidene difluoride (PVDF) membranes (Millipore). The resulting membranes blocked with 5% w/v nonfat milk in Tris-buffered saline (TBS) and probed with 1:10000 rabbit anti-Hsp27 polyclonal antibody (Enzo Life Science, Villeurbanne, France); 1:800 mouse anti-DDX5 monoclonal antibody (sc-365164), 1:500 mouse anti-Ku86 monoclonal antibody (sc-5280), 1:500 mouse anti-Ku70 monoclonal antibody (sc-17789), 1:500 mouse anti-NF45 monoclonal antibody (sc-365283) (Santa Cruz Biotechnology, CA, USA). Vinculin and GAPDH were used as a loading control. Subsequently, the primary Ab was probed with corresponding HRP-conjugated secondary Abs (DAKAO) and detected using ECL prime Western Blotting detection reagent (RPN2236, GE Healthcare).

Results

Elevated DDX5 Protein Expression is Associated with CRPC.

We examined DDX5 expression by immunostaining of a human prostate TMA. Prostate cancer has significantly higher level of DDX5 expression than benign prostatic hyperplasia (BPH). Among 143 PC specimens, Gleason grade 5 prostate cancer has the strongest DDX5 expression, following by Gleason 4 and 3 (data not shown), implying that DDX5 expression is correlated to the aggressiveness of the disease. Moreover, increased DDX5 protein expression was observed in tumors from patients under prolonged neoadjuvant hormone therapy (>6 months) and CRPC patients (data not shown), demonstrating that overexpressed DDX5 is correlated with the progression of CRPC. DDX5 expression was also found to be associated with the metastatic or the stage of prostate cancer since the staining in tumors with lymph node (LN) metastatic was more intensive than in tumors without LN metastatic, and lower in CRPC (data not shown). In addition, the median recurrence free survival (RFS) was 45.4 months in the moderate or strong group, corresponding to a 52% higher relative risk of recurrence compared to the negative or weak group with the median RFS of 76.6 months, indicating that DDX5 overexpression was correlated to a short recurrence free survival (RFS). On the other hand, when it comes to the comparison of DDX5 protein expression between CS (LNCaP) and the CR PC cells (DU145+PC3) by Western Blot, DDX5 expression levels in CR cells are much higher than in the CS cell. Taken together, these results reinforce the point that DDX5 represents a potential target that is relevant in CRPC.

Developing ASOs Targeting DDX5

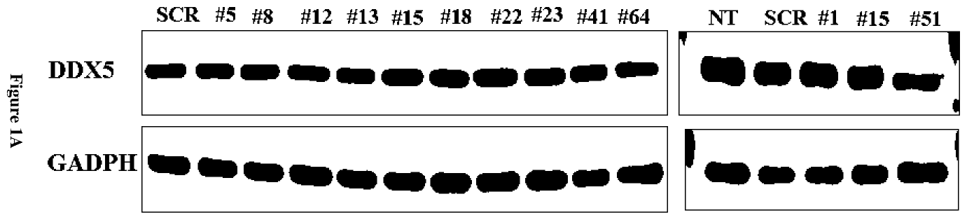

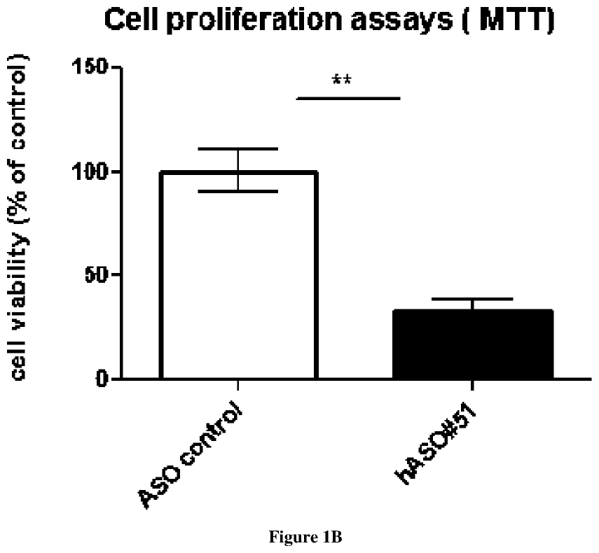

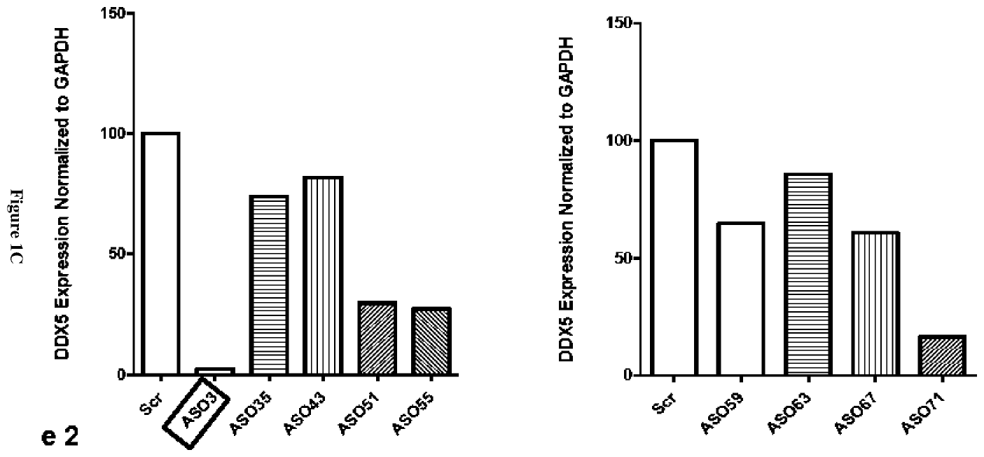

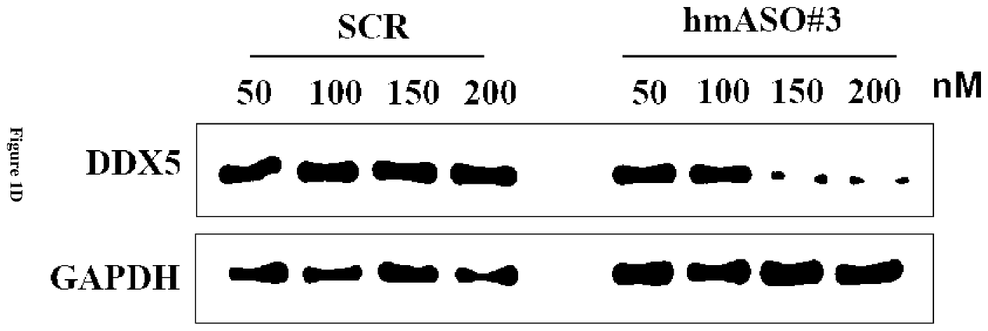

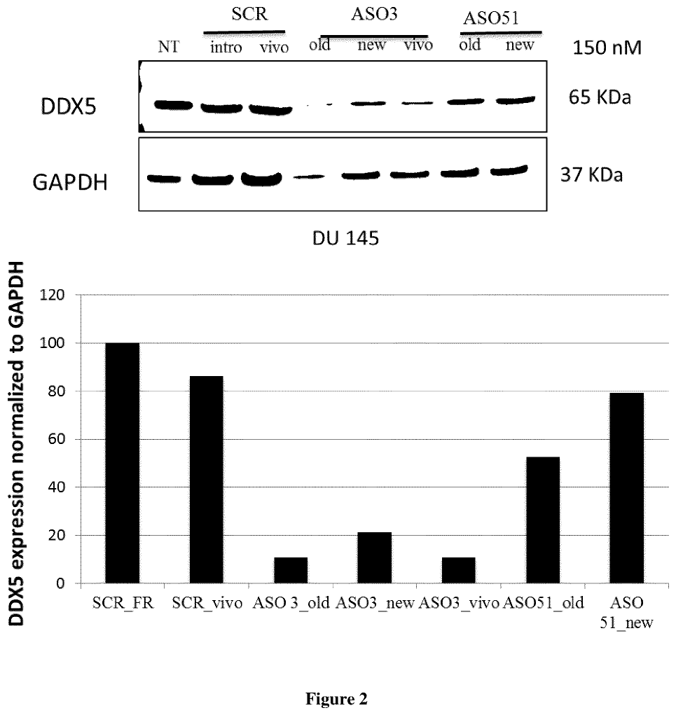

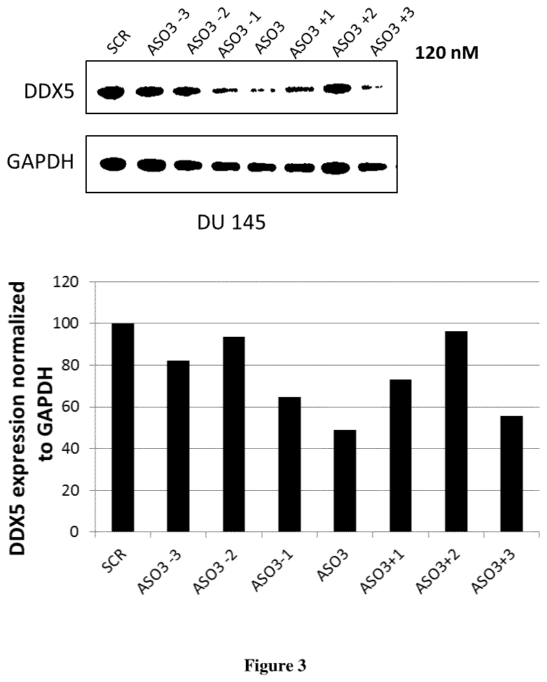

Elevated DDX5 protein expression is correlated to the PC advancement and CRPC progression, so developing a DDX5 inhibitor could be a valuable approach for CRPC treatment. First, the program designed 93 ASOs against the human mRNA sequence of DDX5 (hASO) (data not shown). The 1st screening tested 13 hASOs and showed that hASO #51 decrease DDX5 expression by 81% at 200 nM and in dose-dependent manner (FIG. 1A). Moreover, DDX5 inhibition induced by hASO #51 resulted in a significant decrease in cell viability AI cell line (DU-145) (FIG. 1B). Prior to testing a DDX5 ASO on xenografted mouse models with our human AI cell lines, we continued the screening of DDX5 ASOs to find out a bi-specific sequence of both human and murine form, allowing us to apprehend potential toxic effects of DDX5 inhibition in tumors-carried mice. The percentage of bi-specificity was examined by BLAST analysis of the human and murine form (hmASO), giving 25 bi-specific sequences (data not shown). We tested 8 sequences distributed uniformly over the entire target sequence at different locations of the mRNA in addition to hASO #51. We found that hmASO #3 depleted DDX5 protein expression as effectively as hASO #51 and in a dose-dependent manner (FIGS. 1C and 1D). Therefore, in order to check if DDX5 inhibition is able to restore the tumour sensitivity to CPRC treatments, the hmASO #3 currently becomes the leading DDX5 ASO that is chosen for our in vivo experiment in mice model.

DDX5 is an Hsp27 Partner and Hsp27-Regulated Protein

Hsp27, a small Heat shock protein (sHSP), behaviors as a oncogene during PC progression, its overexpression is positively correlated with metastasis and Castration Resistance emergence (Rocchi et al. 2005). An Antisense Oligonucleotide targeting Hsp27, namely OGX 427, has demonstrated to restore castration and chemotherapy sensitivity of the PC cell. In order to shed light on the mechanism of action in which Hsp27 drives PC initiation and CRPC evolution, several studies which were based on proteomics approaches have been performed in our laboratory. Our previous study aiming to search for the Hsp27- regulated proteins by proteomic profiling comparison between LNCaP-Hsp27 and LNCaP-Mock indicated that DDX5 protein abundance is correlated with Hsp27 protein level (unpublished data). The DDX5 protein level indeed was proved to be higher in the LNCaP-Hsp27 in which Hsp27 gene was stably transfected compared to the LNCaP-Mock cells. Moreover, Hsp27 depletion by OGX-427 decreased significantly DDX5 level in both LNCaP and PC3 (data not shown). Our study on Hsp27 interactome on different PC cell lines using IP/MS suggested that DDX5 is an Hsp27 binding protein (prepared data for submission). The interaction between Hsp27 and DDX5 was confirmed by WB following IP on PC-3 cells (data not shown). All of these results together demonstrated that Hsp27 interacts with DDX5 and regulates DDX5 expression, and Hsp27 might function as a chaperone protein that protects DDX5 from miss folding and ubiquitin-proteasome degradation.

Through Oncomine meta-analysis, DDX5 found to be co-expression with several proteins belonging to the ubiquitin pathway (USP9X, UBE2JI, and UBE3A) and the proteasome (PSMA2, PRKWNK), suggesting the interaction of DDX5 with these proteins and its involvement in these pathways (Wilson and Giguère 2007). Previously, DDX5 was demonstrated to be a target of poly-ubiquitination, hence its stability might be likely regulated through ubiquitin-proteasome system (Causevic et al. 2001) (Mooney et al. 2010). In order to check if the stability of DDX5 is regulated by the 26S proteasome in prostate cancer, the PC3 cells were treated with MG132 at 10 an inhibitor of the proteasome, and with or without cycloheximide (CHX) 10 μg/ml to inhibit the de novo protein synthesis with indicated point of time. Indeed, the DDX5 level increased over course of time due to a blocked proteasome by MG132 (data not shown), and went down by 50%, 70% after inhibiting of protein synthesis 24 hours, 30 hours, respectively (data not shown); meanwhile, it was conserved over time when the PC 3 cells were treated with both MG132, and cycloheximide (CHX) (data not shown). These all suggest that proteasome is likely the main pathway controls the degradation of DDX5 in PC.

Our recent studies have demonstrated that Hsp27 interacts with TCTP (Translationally-controlled tumor protein) and eIF4E (eukaryotic translation initiation factor 4E) and protects these protein partners from proteasome degradation (Andrieu et al. 2010) (Baylot et al. 2012) DDX5 protein levels are elevated in many cancers as mentioned above, but not as a result of upgraded mRNA levels (Causevic et al. 2001). Our DNA microarray data on various models which were up regulated or down regulated of Hsp27 protein expression did not show any difference of the DDX5 mRNA levels among different comparisons such as LNCaP-Hsp27 vs LNCaP-OGX427, LNCaP Hsp27 vs LNCaP, PC3_OGX 427 vs PC3_SCR (data not shown). Indeed, DDX5 expression was confirmed to not be controlled at the transcription level by Hsp27 (data not shown), suggesting that Hsp27 may control DDX5 protein stability. To identify how Hsp27 modulates DDX5 abundance, the DDX5 half-life was determined after OGX 427 treatment. The PC3 cells were transfected with ASO of Hsp27, OGX427 or the ASO control and treated with or without MG132/CHX for 48 hours before being harvested for protein extractions. MG132/CHX treatment extents DDX5 half-life and reversed the effect of Hsp27 depletion by ASO OGX-427, suggesting that DDX5 depletion after OGX427-induced Hsp27 knockdown is as a result of proteasome degradation (data not shown)

In order to know if there is any feedback loop between 2 proteins, we checked the Hsp27 abundance after DDX5 depletion. We did not observe any changes of the Hsp27 protein level due to DDX5 inhibition (data not shown). Therefore, it is likely that Hsp27 controls the DDX5 stability and DDX5 does not regulate Hsp27 expression.

DDX5 Promotes CRPC by Activating AKT/mTOR1 Pathway

DDX5 was demonstrated to promote cell survival and growth by activating the mTORC1 signaling pathway (Taniguchi et al. 2016). DDX5 was previously showed to control p-S6K1 and p 4E BP1 levels, two well-known effectors of activated mTOR signaling. We examined if DDX5 can regulate the upstream components belonging to the pathway such as mTOR1, AKT. We found that DDX5 inhibition significantly decreased protein levels of both AKT and p-AKT and p-mTOR (data not shown).

Identification of DDX5 Interactomes in Various PC Cell Lines by IP Coupled LC-MS/MS

To shed light on the mechanism of actions by which DDX5 drives CRPC progression, we identified the PPI (protein-protein interaction) networks of DDX5 in four PC cell lines that present for different stages of PC progression (data not shown). The protein complexes of endogenous DDX5 containing its binding proteins were captured by immunoprecipitation (IP) using the antibody against DDX5 and subsequently determined by LS/MS/MS. The software MaxQuant was applied to remove nonspecific binding proteins and give the list of DDX5-associated proteins (data not shown). The silver staining analyses 10% of the IP elution showed the band of DDX5 on the IP samples but not on the controls, which illustrated that our IP experiment works very well (data not shown). Three biological replicates were performed along with 3 technical triplicate of LC-MS/MS and gained very high reproducibility (correlation value <8e-1) for all the cell lines (data not shown). In total, 489 proteins were identified, and we obtained 16, 44,239 and 401 candidates for PNT1a, LNCaP, DU 145, and PC-3 respectively (data not shown). We also classified the proteins found by IP/MS into 2 classes: high evidence (present in both filters FDR 0.1 and FDR=1) and the 2 one is just satisfactory FDR=1 but not FDR=0.1. Obviously, an association of the number of DDX5 binding proteins with the aggressiveness of the disease and CRPC was observed, this indicates that DDX5 might extent more cellular functions during the disease development. The Venny diagram showed a cross among 4 lines ‘datasets (data not shown). 173 candidates are shared between 2 CR lines (DU 145 and PC 3), which covered up to 72.3% of the interactors determined in DU 145 (data not shown). The PPI interaction network of the 489 proteins obtained in four cell lines which were constructed using the STRING database showed a very high number of connections among 487 nodes (487/489, 99.9%) with 13522 edges (average node degree: 55.5, avg. local clustering coefficient: 0.54, PPI enrichment p-value: <1.0e-16). DDX5 is connected to 113 proteins (113/487, approximately 23.2%) in the network, (data not shown). Especially, we found 59/113 proteins inside the network which were considered to have known interactions with DDX5 from both experimentally determined and curated databases. DDX17 which is known as DDX5 paralog, and well-described interactor of DDX5 were found in all of 4 cell line dataset, and in the network with combined score 0.75 (Ogilvie et al. 2003). In addition, we also obtained TP53, DHX9,CDK9, which are very well described as DDX5 partners (Bates et al. 2005) (Nicol et al. 2013a) (Wilson and Giguère 2007) (Yang et al. 2015). A number of proteins have very high combined score (above 0.9) such as HNRNPL, ELAVL1, FUS, HNRNPA0, SRSF1, and YBX1; and so on (data not shown). These all together proved the highly efficiency of our Co-IP-LC-MS/MS.

Characterization of the Global DDX5 Interactome in PC

The DDX5-interacting proteins found in 4 cell lines was functional classified by using PANTHER 14.1. When it comes to Molecular function, nearly a half of the DDX5 interactome involve in “binding” in which they interact with other molecules such as: nucleic acid, protein, protein containing complex, and chromatin. A significant number of the DDX5 partners possess enzyme activity (catalytic activity: 22%) and contribute to complex assembly (structural molecule activity: 20%) (data not shown). In the consistent with these, classifying the DDX5 interactome based on the PANTHER “protein class” database showed that a majority of the DDX5 interactors are involved in binding to nucleic acid and most of them are RNA binding proteins (data not shown). It also revealed several protein classes engaging with catalytic functions such as hydrolase (9.2%), transferase (8.1%), ligase (1.4%), and enzyme modulator (4.6%). Interestingly, we could recognize a set of DDX5-associated proteins which function as transcription factors (23 proteins, 6.6%) and 8 of them are zinc finger transcription factors.

In order to decipher the putative functions associated with DDX5-interacting proteins in PC, we performed the analysis of Gene Ontology (GO) enrichment using BiNGO tool run by Cytoscape (Maere et al. 2005) for the set of total DDX5 interactors found in 4 cell lines, and 126, and 79 functions were obtained with significant level p=0.05 and 0.005, respectively (data not shown). As expected, well-known key functions of DDX5 were observed. We found that DDX5 plays vital roles in gene expression since a majority of DDX5 interactors (up to 57%) are engaged with this process (p=0.0000E-100). DDX5 functions mainly in RNA processing (p=1.2751E-72), translation (p=1.8605E-71), ribosome biogenesis (p=1.8027E-42) and transcription (p=2.8988E-6). In addition, DDX5 likely modulates gene expression at posttranscriptional levels by regulating mRNA stability, RNA splicing and translation. On the other hand, we found that DDX5 participates in the cellular response to the DNA damage stimulus (p=4.4587E-3) including DNA repair process. Beside involving to the p53 related-DNA damage response as described before (Nicol et al. 2013b), we found novel functions of DDX5 in DNA repair pathways such as: Non homologous end-joining (NHEJ) and Nucleotide excision repair (NER).

By performing a functional enrichment analysis using Gprofiler (Raudvere et al. 2019) for our DDX5 associated proteins with the CORUM 3.0 database (https://mips.helmholtz-muenchen.de/corum/) which collects annotation of mammalian protein complexes obtained from manual experiments (Giurgiu et al. 2019), we determined various protein complexes corresponding to different functions in the DDX5 interactome (data not shown). Consistently, we found that DDX5 tightly associates with the complexes involved in ribosome biogenesis, protein synthesis in cytoplasm and mitochondria, splicing, mRNA stability, transcription and DNA repair.

DDX5 likely modulates the mRNA stability via interacting with IGF2BPs complex which is consisted of 9 proteins: RPS6, RPL26, DHX9, STAU1, ELAVL1, SYNCRIP, HNRNPU, IGF2BPs (IGF2BP1,IGF2BP2, IGF2BP3), and YBX1 (Weidensdorfer et al. 2009). The main function of IGF2BPs complex is to enhance the stability and storage of the target mRNA by associating with the Coding Region instability Determinant (CRD) (Huang et al. 2018), including IGF2 (Dai et al. 2017) (Cao et al. 2018), MYC (Noubissi et al. 2006) (Weidensdorfer et al. 2009), ACTIN (Hüttelmaier et al. 2005) and LIN28B (Hafner et al. 2010). Moreover, DHX9, IGF2BPs and YBX1 have been proved to play oncogenic function and therapy resistant in various cancers. DDX5 tightly associates with the IGF2BP2 and IGF2BP3 complex since we found all of their protein components in our DDX5 interactome (data not shown). Moreover, DDX5 has been showed to be associated with DHX9, STAU1, ELAVL1, SYNCRIP, HNRNPU, IGF2BP3, and YBX1 based on string database.

The identified DDX5 interactome showed a solid connection between DDX5 and toposome, revealing novel mechanism of action by which DDX5 regulates cell cycle. Toposome which consists of TOP2A, SRPK1, DHX9, HNRNPC, PRPF8, DDX21, and SSRP1 plays essential roles in cell cycle regulation by modeling chromosome segregation, chromosome topological changes (Lee et al. 2004). Except SSRP1 found to interact with DDX5 in database, all of proteins belonging to Toposome were determined to associate with DDX5 in our study. We first introduced the interaction of DDX5 and toposome, especially TOP2A, a widespread drug target in many types of cancer (Nitiss 2009) (Pogorelcnik and Solmajer 2013). Moreover, our study also confirmed the interaction between DDX5 and TOP1, which was annotated in the string database. This can provide a clue about the mechanism by which DDX5 overexpression conferred to resistance to Camptothecin (CPT), the inhibitor of TOP1 (Cohen et al. 2008).

Interestingly, we first showed the rigid association of DDX5 with the transcription factor complex TH2H which is composed of the core complex (GTF2H1, GTF2H2, GTF2H3, GTF2H4, ERCC2, ERCC3) and the CAK (CDK7, CCNH, MNAT1) (data not shown). The TH2H complex functions in both transcription and DNA damage response, suggesting the involvement of DDX5 in these biological processes.

Another novel mechanism in which DDX5 modulates transcription is through the 7SK RPN complex. All the proteins constructing to the complex are found to associate with DDX5 in our study, including CDK9, HEXIM1, CCNT1, and LARP7. The positive transcription elongation factor B, p-TEFb which is composed of CDK9 and CCNT1 is tightly inactivated by its association with LARP7, HEXIM1 and 7SK. Activated P-TEFb is recruited to the initiation complex by interacting with Transcription factors and phosphorylates CTD Ser2 for effective elongation (Romano 2013) (Rahaman et al. 2016).Especially, p-TEFb was shown to be activated by PSA eRNA by which it regulates AR targeted genes transcription in CRPC, promoting Castration resistance emergency (Zhao et al. 2016). The significant of the association between DDX5 and the 7SK complex and p-TDFb is uncharacterized, it calls for further investigation.

DDX5 Promotes CRPC by Regulation of DNA Damage Response

By using the ClusterProfiler R packages (Yu et al. 2012) we compared the Gene Ontology Biological Functions (GO BP) among different DDX5 interactomes found in NM, CS, CR cells. The results revealed that DDX5 involves in much more biological functions in CR cells, which mainly related to DNA damage response, translation, transcription, RNA stability, and DNA conformation changes (data not shown). It is believed that DDX5 can promote CRPC development by its participation in these set of exclusive functions found in CR cells.

It worth noting that we determined a number of novel DDX5 binding proteins involved in DNA repair and exclusively found in the CR lines, uncovering new potential roles of DDX5 in DNA damage response, genomic integrity and CRPC development. The proteins belonging to DNA repair function relied on the BinGO analyses in DU 145 and PC 3 cells are 16 and 15, respectively (data not shown). The main common DNA repair pathways found in both CR cell lines are NHEJ (NHEJ-dependent DSBR) and NER (data not shown). Visualization of these two pathways by KEGG database via Pathview by R showed the DDX5 interactors involved in NHEJ and NER mechanisms (data not shown). The PPI network of DDX5 and 20 proteins belonging to DNA repair module was generated by STRING, and DDX5 was showed to have known interaction with 3 proteins: P53, UPF1, and PRP19. This means that our approach allowed us to discover novel DDX5 binding proteins which participate in DDR, revealing new potential functions of DDX5 in two DNA repair pathways, NHEJ (via Ku complex) and NER (via GTF2H complex).

Our IP/MS analyses indicated that DDX5 is associated with the Ku70/ku86 complex. We confirmed the binding of DDX5 and the Ku70/ku86 complex by WB followed IP. Ku 70, Ku 86 and NF45 (ILF2) present in the IP using the Ab against DDX5 on both cell lines DU 145 and PC 3 (data not shown). On the other hand, DDX5 is found in the Reverse IP (RIP) with Ku70 and Ku86 Ab (data not shown). These prove the interaction of DDX5 with the core complex of NHEJ, Ku70/Ku86.

To shed light on DDX5 potential functions in DNA damage repair, we examined how knock out DDX5 affects recovery of DNA repair. The DU 145 cells were transfected with SCR-ASO as a control and ASO51 to induce DDX5 depletion, and objected to irradiation after 72h of transfection. The dynamic of DNA damage recovery was analyzed by IF using anti-p gH2AX foci over time-points: 0 hr, 0.25 hr, 1 hr, 3 hr, 6h, and 24 hr. The foci numbers in the DDX5-depleted cells were lower than those in the control samples from 3 hrs to 24 hrs (data not shown). This implied that DDX5 knock down enhanced efficiency of DNA repair recovery. In another words, DDX5 negatively regulated DNA damage repair.

ASO3 Downregulates DDX5 Protein Expression Highly Effectively.

Different batches of ASO3 and ASO51 was then tested for their ability to downregulate DDX5 expression. The results confirmed that ASO3 downregulates DDX5 protein expression highly effectively and even better than the ASO51 in DU-145 cells (FIG. 2). While using ASO3 at 150 nM induced around 80% DDX5 inhibition (FIG. 2), it caused too much death cells making it difficult to obtain protein extract. Basing on this results, ASO3 and nearby ASO3 at 120 nM are tested. The results showed that ASO3-1, ASO3+1 and ASO3+3 also inhibit DDX5 protein level beside the ASO3 (FIG. 3). From all these results, it is concluded that ASO3 is the one which can inhibit DDX5 expression more effectively than other tested ASOs in DU-145 cells.

### CONCLUSION

DDX5 has previously described to be involved in DNA damage response (Nicol et al. 2013b). In this publication, DDX5 was shown to recruit both p53 and RNAPII to the p21 promoter upon irradiation stress, resulting in cell cycle arrest after DNA damage. The study of the inventors has demonstrated novel mechanism of actions of DDX5 involving in DNA damage response in prostate cancer. DDX5 is likely the central of different DNA repair pathways, such as NHEJ, NER. They have proved that depleted DDX5 enhances significantly DNA damage recovery. In another words, DDX5 negatively regulates DNA repair process, providing more advantages for the survival of the tumor cells upon DNA damage-caused stresses such as chemotherapy or irradiation.

### REFERENCES

Throughout this application, various references describe the state of the art to which this invention pertains. The disclosures of these references are hereby incorporated by reference into the present disclosure.
Andrieu C, Taieb D, Baylot V, et al (2010) Heat shock protein 27 confers resistance to androgen ablation and chemotherapy in prostate cancer cells through eIF4E. Oncogene 29:1883-1896. https://doi.org/10.1038/onc.2009.479
Bates G J, Nicol S M, Wilson B J, et al (2005) The DEAD box protein p68: a novel transcriptional coactivator of the p53 tumour suppressor. EMBO J 24:543-553. https://doi.org/10.1038/sj.emboj.7600550
Baylot V, Katsogiannou M, Andrieu C, et al (2012) Targeting TCTP as a new therapeutic strategy in castration-resistant prostate cancer. Mol Ther J Am Soc Gene Ther 20:2244-2256. https://doi.org/10.1038/mt.2012.155
Bond A T, Mangus D A, He F, Jacobson A (2001) Absence of Dbp2p alters both nonsense-mediated mRNA decay and rRNA processing. Mol Cell Biol 21:7366-7379. https://doi.org/10.1128/MCB.21.21.7366-7379.2001
Cao J, Mu Q, Huang H (2018) The Roles of Insulin-Like Growth Factor 2 mRNA-Binding Protein 2 in Cancer and Cancer Stem Cells. Stem Cells Int 2018: https://doi.org/10.1155/2018/4217259
Caretti G, Schiltz R L, Dilworth F J, et al (2006) The RNA helicases p68/p72 and the noncoding RNA SRA are coregulators of MyoD and skeletal muscle differentiation. Dev Cell 11:547-560. https://doi.org/10.1016/j.devce1.2006.08.003
Causevic M, Hislop R G, Kernohan N M, et al (2001) Overexpression and poly-ubiquitylation of the DEAD-box RNA helicase p68 in colorectal tumours. Oncogene 20:7734. https://doi.org/10.1038/sj.onc.1204976
Chevallet M, Luche S, Rabilloud T (2006) Silver staining of proteins in polyacrylamide gels. Nat Protoc 1:1852-1858. https://doi.org/10.1038/nprot.2006.288
Chi K N, Yu E Y, Jacobs C, et al (2016) A phase I dose-escalation study of apatorsen (OGX-427), an antisense inhibitor targeting heat shock protein 27 (Hsp27), in patients with castration-resistant prostate cancer and other advanced cancers. Ann Oncol 27:1116-1122. https://doi.org/10.1093/annonc/mdw068
Choi S Y C, Xue H, Wu R, et al (2016) The MCT4 Gene: A Novel, Potential Target for Therapy of Advanced Prostate Cancer. Clin Cancer Res Off J Am Assoc Cancer Res 22:2721-2733. https://doi.org/10.1158/1078-0432.CCR-15-1624
Clark E L, Coulson A, Dalgliesh C, et al (2008) The RNA helicase p68 is a novel androgen receptor coactivator involved in splicing and is overexpressed in prostate cancer. Cancer Res 68:7938-7946. https://doi.org/10.1158/0008-5472.CAN-08-0932
Clark E L, Hadjimichael C, Temperley R, et al (2013) p68/DdX5 Supports β-Catenin & RNAP II during Androgen Receptor Mediated Transcription in Prostate Cancer. PLOS ONE 8:e54150. https://doi.org/10.1371/journal.pone.0054150
Cohen A A, Geva-Zatorsky N, Eden E, et al (2008) Dynamic proteomics of individual cancer cells in response to a drug. Science 322:1511-1516. https://doi.org/10.1126/science.1160165
Dai N, Ji F, Wright J, et al IGF2 mRNA binding protein-2 is a tumor promoter that drives cancer proliferation through its client mRNAs IGF2 and HMGA1 eLife 6: https://doi.org/10.7554/eLife.27155
Dardenne E, Pierredon S, Driouch K, et al (2012) Splicing switch of an epigenetic regulator by RNA helicases promotes tumor-cell invasiveness. Nat Struct Mol Biol 19:1139-1146. https://doi.org/10.1038/nsmb.2390
Fuller-Pace F V (2006) DExD/H box RNA helicases: multifunctional proteins with important roles in transcriptional regulation. Nucleic Acids Res 34:4206-4215. https://doi.org/10.1093/nar/gkl460
Guturi K K N, Sarkar M, Bhowmik A, et al (2014) DEAD-box protein p68 is regulated by β-catenin/transcription factor 4 to maintain a positive feedback loop in control of breast cancer progression. Breast Cancer Res BCR 16: https://doi.org/10.1186/s13058-014-0496-5
Hafner M, Landthaler M, Burger L, et al (2010) Transcriptome-wide identification of RNA-binding protein and microRNA target sites by PAR-CLIP. Cell 141:129-141. https://doi.org/10.1016/j.ce11.2010.03.009
Huang H, Weng H, Sun W, et al (2018) Recognition of RNA N6-methyladenosine by IGF2BP proteins enhances mRNA stability and translation. Nat Cell Biol 20:285-295. https://doi.org/10.1038/s41556-018-0045-z
Hüttelmaier S, Zenklusen D, Lederer M, et al (2005) Spatial regulation of beta-actin translation by Src-dependent phosphorylation of ZBP1. Nature 438:512-515. https://doi.org/10.1038/nature04115
Jalal C, Uhlmann-Schiffler H, Stahl H (2007) Redundant role of DEAD box proteins p68 (Ddx5) and p72/p82 (Ddx17) in ribosome biogenesis and cell proliferation. Nucleic Acids Res 35:3590-3601. https://doi.org/10.1093/nar/gkm058
Janknecht R (2010) Multi-talented DEAD-box proteins and potential tumor promoters: p68 RNA helicase (DDX5) and its paralog, p72 RNA helicase (DDX17). Am J Transl Res 2:223-234
Karaki S, Paris C, Rocchi P (2019) Antisense Oligonucleotides, A Novel Developing Targeting Therapy. Antisense Ther. https://doi.org/10.5772/intechopen.82105
Katsogiannou M, Ziouziou H, Karaki S, et al (2015) The hallmarks of castration-resistant prostate cancers. Cancer Treat Rev 41:588-597. https://doi.org/10.1016/j.ctrv.2015.05.003
Lee C-G, Hague L K, Li H, Donnelly R (2004) Identification of toposome, a novel multisubunit complex containing topoisomerase IIalpha. Cell Cycle Georget Tex 3:638-647
Lin S, Tian L, Shen H, et al (2013) DDX5 is a positive regulator of oncogenic NOTCH1 signaling in T cell acute lymphoblastic leukemia. Oncogene 32:4845-4853. https://doi.org/10.1038/onc.2012.482
Ma Z, Feng J, Guo Y, et al (2017) Knockdown of DDX5 Inhibits the Proliferation and Tumorigenesis in Esophageal Cancer. Oncol Res 25:887-895. https://doi.org/10.3727/096504016X14817158982636
Mazurek A, Luo W, Krasnitz A, et al (2012) DDX5 regulates DNA replication and is required for cell proliferation in a subset of breast cancer cells. Cancer Discov 2:812-825. https://doi.org/10.1158/2159-8290.CD-12-0116
Mooney S M, Grande J P, Salisbury J L, Janknecht R (2010) Sumoylation of p68 and p72 RNA helicases affects protein stability and transactivation potential. Biochemistry 49:1-10. https://doi.org/10.1021/bi901263m
Nicol S M, Bray S E, Black H D, et al (2013a) The RNA helicase p68 (DDX5) is selectively required for the induction of p53-dependent p21 expression and cell-cycle arrest after DNA damage. Oncogene 32:3461-3469. https://doi.org/10.1038/onc.2012.426
Nicol S M, Bray S E, Black H D, et al (2013b) The RNA helicase p68 (DDX5) is selectively required for the induction of p53-dependent p21 expression and cell cycle arrest after DNA damage. Oncogene 32:3461-3469. https://doi.org/10.1038/onc.2012.426
Nyamao R M, Wu J, Yu L, et al (2019) Roles of DDX5 in the tumorigenesis, proliferation, differentiation, metastasis and pathway regulation of human malignancies. Biochim Biophys Acta Rev Cancer 1871:85-98. https://doi.org/10.1016/j.bbcan.2018.11.003
Ogilvie V C, Wilson B J, Nicol S M, et al (2003) The highly related DEAD box RNA helicases p68 and p72 exist as heterodimers in cells. Nucleic Acids Res 31:1470-1480. https://doi.org/10.1093/nar/gkg236
Pogorel_cnik B, Solmajer A P and T (2013) Recent Advances in the Development of Catalytic Inhibitors of Human DNA Topoisomerase IIα_As Novel Anticancer Agents. In: Curr. Med. Chem. http://www.eurekaselect.com/106834/article. Accessed 7 Oct. 2019
Rahaman M H, Kumarasiri M, Mekonnen L B, et al (2016) Targeting CDK9: a promising therapeutic opportunity in prostate cancer. Endocr Relat Cancer 23:T211-T226. https://doi.org/10.1530/ERC-16-0299
Rinaldi C, Wood M J A (2018) Antisense oligonucleotides: the next frontier for treatment of neurological disorders. Nat Rev Neurol 14:9-21. https://doi.org/10.1038/nrneuro1.2017.148
Rocchi P, Beraldi E, Ettinger S, et al (2005) Increased Hsp27 after Androgen Ablation Facilitates Androgen-Independent Progression in Prostate Cancer via Signal Transducers and Activators of Transcription 3-Mediated Suppression of Apoptosis. Cancer Res 65:11083-11093. https://doi.org/10.1158/0008-5472.CAN-05-1840
Rocchi P, Jugpal P, So A, et al (2006) Small interference RNA targeting heat-shock protein 27 inhibits the growth of prostatic cell lines and induces apoptosis via caspase-3 activation in vitro. BJU Int 98:1082-1089. https://doi.org/10.1111/j.1464-410X.2006.06425.x
Rocchi P, So A, Kojima S, et al (2004) Heat shock protein 27 increases after androgen ablation and plays a cytoprotective role in hormone-refractory prostate cancer. Cancer Res 64:6595-6602. https://doi.org/10.1158/0008-5472.CAN-03-3998
Romano G (2013) Deregulations in the Cyclin-Dependent Kinase-9-Related Pathway in Cancer: Implications for Drug Discovery and Development. In: Int. Sch. Res. Not. https://www.hindawi.com/journals/isrn/2013/305371/. Accessed 27 Jun. 2019
Rosenberg J E, Hahn N M, Regan M M, et al (2018) Apatorsen plus docetaxel versus docetaxel alone in platinum-resistant metastatic urothelial carcinoma (Borealis-2). Br J Cancer 118:1434-1441. https://doi.org/10.1038/s41416-018-0087-9
Shin S, Rossow K L, Grande J P, Janknecht R (2007) Involvement of RNA helicases p68 and p72 in colon cancer. Cancer Res 67:7572-7578. https://doi.org/10.1158/0008-5472.CAN-06-4652
Taniguchi T, Iizumi Y, Watanabe M, et al (2016) Resveratrol directly targets DDX5 resulting in suppression of the mTORC1 pathway in prostate cancer. Cell Death Dis 7:e2211. https://doi.org/10.1038/cddis.2016.114
Thomas C, Zoubeidi A, Kuruma H, et al (2011) Transcription factor StatS knockdown enhances androgen receptor degradation and delays castration-resistant prostate cancer progression in vivo. Mol Cancer Ther 10:347-359. https://doi.org/10.1158/1535-7163.MCT-10-0850
Wang Z, Luo Z, Zhou L, et al (2015) DDX5 promotes proliferation and tumorigenesis of non-small-cell lung cancer cells by activating β-catenin signaling pathway. Cancer Sci 106:1303-1312. https://doi.org/10.1111/cas.12755
Weidensdorfer D, Stohr N, Baude A, et al (2009) Control of c-myc mRNA stability by IGF2BP1-associated cytoplasmic RNPs. RNA N Y N 15:104-115. https://doi.org/10.1261/rna.1175909
Wilson B J, Giguère V (2007) Identification of novel pathway partners of p68 and p72 RNA helicases through Oncomine meta-analysis. BMC Genomics 8:419. https://doi.org/10.1186/1471-2164-8-419
Yang J, Zhao Y, Kalita M, et al (2015) Systematic Determination of Human Cyclin Dependent Kinase (CDK)-9 Interactome Identifies Novel Functions in RNA Splicing Mediated by the DEAD Box (DDX)-5/17 RNA Helicases. Mol Cell Proteomics 14:2701-2721. https://doi.org/10.1074/mcp.M115.049221
Yang L, Lin C, Liu Z-R (2006) P68 RNA helicase mediates PDGF-induced epithelial mesenchymal transition by displacing Axin from beta-catenin. Cell 127:139-155. https://doi. org/10.1016/j. cell.2006.08.036
Yu E Y, Ellard S L, Hotte S J, et al (2018) A randomized phase 2 study of a HSP27 targeting antisense, apatorsen with prednisone versus prednisone alone, in patients with metastatic castration resistant prostate cancer. Invest New Drugs 36:278-287. https://doi.org/10.1007/s10637-017-0553-x
Yu G, Wang L-G, Han Y, He Q-Y (2012) clusterProfiler: an R Package for Comparing Biological Themes Among Gene Clusters. OMICS J Integr Biol 16:284-287. https://doi.org/10.1089/omi.2011.0118
Zhao Y, Wang L, Ren S, et al (2016) Activation of P-TEFb by Androgen Receptor-Regulated Enhancer RNAs in Castration-Resistant Prostate Cancer. Cell Rep 15:599-610. https://doi.org/10.1016/j.celrep.2016.03.038

1. A method of treating cancer in a subject in need thereof, comprising administering to the subject a therapeutically effective amount of an inhibitor of DDX5, wherein said inhibitor reduces expression and/or activity of DDX5 and targets the gene or mRNA of DDX5.

2. The method according to claim 1 wherein said inhibitor reduces the expression and/or the activity of DDX5 and targets the nucleic acids sequence SEQ ID NO: 94.

3. The method according to claim 1 wherein said inhibitor reduces the expression and/or the activity of DDX5 and targets at least from 15 to 25 nucleic acids of SEQ ID NO: 94.

4. The method according to claim 1 wherein said inhibitor reduces the expression and/or the activity of DDX5 and targets at least a region comprising nucleic acids 276-515, or 1056-1155 or 1396-1795 or 1856-1955 of SEQ ID NO: 94.

5. The method according to claim 1 wherein said inhibitor is a siRNA, a shRNA, an antisense oligonucleotide, miRNA or a ribozyme.

6. The method according to claim 3 wherein said inhibitor is an antisense oligonucleotide selected from the group consisting of: SEQ ID NO:1 to SEQ ID NO:93.

7. The method according to claim 5 wherein said inhibitor is an antisense oligonucleotide set forth as SEQ ID NO:3.

8. A vector comprising a heterologous nucleic acid, wherein the heterologous nucleic acid encodes an inhibitor according to claim 1.

9. (canceled)

10. The method according to claim 1, wherein the cancer is selected from the group consisting of adrenal cortical cancer, anal cancer, bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

11. The method according to claim 1, wherein the cancer is a prostate cancer, a resistant prostate cancer or a Castration Resistant Prostate Cancer (CRPC).

12. A pharmaceutical composition which comprises an inhibitor according to claim 1.

13. (canceled)

14. The method according to claim 9, wherein the cancer is a chemotherapy or radiotherapy resistant prostate cancer.


SEQUENCE LISTING (95 sequences)

SEQ ID NO: 1 - DNA, 20 bp, Artificial
  Synthetic ASO 1
tcactcgaataacccgacat

SEQ ID NO: 2 - DNA, 20 bp, Artificial
  Synthetic ASO 2
ggtcccggccgcggtctcgg

SEQ ID NO: 3 - DNA, 20 bp, Artificial
  Synthetic ASO 3
tcgcggtgcaccaaaccctc

SEQ ID NO: 4 - DNA, 20 bp, Artificial
  Synthetic ASO 4
cctgccctacttcctccaaa

SEQ ID NO: 5 - DNA, 20 bp, Artificial
  Synthetic ASO 5
acttctttccagataagggc

SEQ ID NO: 6 - DNA, 20 bp, Artificial
  Synthetic ASO 6
tttctccccagggtttccaa

SEQ ID NO: 7 - DNA, 20 bp, Artificial
  Synthetic ASO 7
ttccacttctttttaactaa

SEQ ID NO: 8 - DNA, 20 bp, Artificial
  Synthetic ASO 8
atttaggcagctcatcaaga

SEQ ID NO: 9 - DNA, 19 bp, Artificial
  Synthetic ASO 9
ttgataaaattcttctcaa

SEQ ID NO: 10 - DNA, 20 bp, Artificial
  Synthetic ASO 10
ctagccaaatcagggtgctc

SEQ ID NO: 11 - DNA, 20 bp, Artificial
  Synthetic ASO 11
ccacctcttgtgctgtgcgc

SEQ ID NO: 12 - DNA, 20 bp, Artificial
  Synthetic ASO 12
cttgcttcttctgtatgttt

SEQ ID NO: 13 - DNA, 20 bp, Artificial
  Synthetic ASO 13
tgacctctaactgtaatttc

SEQ ID NO: 14 - DNA, 20 bp, Artificial
  Synthetic ASO 14
gaactggcttcgggcagttg

SEQ ID NO: 15 - DNA, 20 bp, Artificial
  Synthetic ASO 15
attggcttcataaaaattta

SEQ ID NO: 16 - DNA, 20 bp, Artificial
  Synthetic ASO 16
tccatgacatttgcagggaa

SEQ ID NO: 17 - DNA, 20 bp, Artificial
  Synthetic ASO 17
aattctgtcttgcaataaca

SEQ ID NO: 18 - DNA, 20 bp, Artificial
  Synthetic ASO 18
aatacgagtgggttcagtga

SEQ ID NO: 19 - DNA, 20 bp, Artificial
  Synthetic ASO 19
actggccatccctgtgcttg

SEQ ID NO: 20 - DNA, 21 bp, Artificial
  Synthetic ASO 20
tatccaatcccacttagagca

SEQ ID NO: 21 - DNA, 20 bp, Artificial
  Synthetic ASO 21
agtctgtgccactccaacca

SEQ ID NO: 22 - DNA, 20 bp, Artificial
  Synthetic ASO 22
gacaatgttttcccagatcc

SEQ ID NO: 23 - DNA, 20 bp, Artificial
  Synthetic ASO 23
caatggcaggaagcaaataa

SEQ ID NO: 24 - DNA, 20 bp, Artificial
  Synthetic ASO 24
tggctgatgattgatgtgga

SEQ ID NO: 25 - DNA, 20 bp, Artificial
  Synthetic ASO 25
ccatcgcctctctctaggaa

SEQ ID NO: 26 - DNA, 20 bp, Artificial
  Synthetic ASO 26
ccagcaccaaacaaataggc

SEQ ID NO: 27 - DNA, 20 bp, Artificial
  Synthetic ASO 27
ggccagttcccgagttggtg

SEQ ID NO: 28 - DNA, 20 bp, Artificial
  Synthetic ASO 28
gctacttgctgcacctgttg

SEQ ID NO: 29 - DNA, 20 bp, Artificial
  Synthetic ASO 29
atgctctacaatattcacga

SEQ ID NO: 30 - DNA, 19 bp, Artificial
  Synthetic ASO 30
acaagtagattcaagcgac

SEQ ID NO: 31 - DNA, 20 bp, Artificial
  Synthetic ASO 31
ttaggagcaccaccgtagat

SEQ ID NO: 32 - DNA, 20 bp, Artificial
  Synthetic ASO 32
aatcacgtatttgtggtccc

SEQ ID NO: 33 - DNA, 19 bp, Artificial
  Synthetic ASO 33
gatttcctcattctctcca

SEQ ID NO: 34 - DNA, 20 bp, Artificial
  Synthetic ASO 34
cttccaggtgttgcaataca

SEQ ID NO: 35 - DNA, 20 bp, Artificial
  Synthetic ASO 35
actctaaaaagtcaatcagt

SEQ ID NO: 36 - DNA, 20 bp, Artificial
  Synthetic ASO 36
tctcagattggtttttccac

SEQ ID NO: 37 - DNA, 20 bp, Artificial
  Synthetic ASO 37
aggacaaggtaggttgttct

SEQ ID NO: 38 - DNA, 20 bp, Artificial
  Synthetic ASO 38
gcattctatctgcttcatca

SEQ ID NO: 39 - DNA, 20 bp, Artificial
  Synthetic ASO 39
gggttcaaagcccatatcaa

SEQ ID NO: 40 - DNA, 20 bp, Artificial
  Synthetic ASO 40
tccacaatcttccttatttg

SEQ ID NO: 41 - DNA, 20 bp, Artificial
  Synthetic ASO 41
gcctatcaggtcttatttga

SEQ ID NO: 42 - DNA, 20 bp, Artificial
  Synthetic ASO 42
cgcactccacattagagttt

SEQ ID NO: 43 - DNA, 20 bp, Artificial
  Synthetic ASO 43
cttacttctttcggccaagt

SEQ ID NO: 44 - DNA, 20 bp, Artificial
  Synthetic ASO 44
ggaaatcttcagcaagctgt

SEQ ID NO: 45 - DNA, 20 bp, Artificial
  Synthetic ASO 45
tatatgaatatagtctttca

SEQ ID NO: 46 - DNA, 20 bp, Artificial
  Synthetic ASO 46
agttcaagtgcaccaatgtt

SEQ ID NO: 47 - DNA, 20 bp, Artificial
  Synthetic ASO 47
gaatgttgtggtttgcactc

SEQ ID NO: 48 - DNA, 20 bp, Artificial
  Synthetic ASO 48
acacacatccacaatgtgaa

SEQ ID NO: 49 - DNA, 20 bp, Artificial
  Synthetic ASO 49
tcatccttttctacgtcatg

SEQ ID NO: 50 - DNA, 20 bp, Artificial
  Synthetic ASO 50
ccattagacgaataagtttt

SEQ ID NO: 51 - DNA, 20 bp, Artificial
  Synthetic ASO 51
cttctcactcatgatctctt

SEQ ID NO: 52 - DNA, 20 bp, Artificial
  Synthetic ASO 52
aaaacaatggttttattctc

SEQ ID NO: 53 - DNA, 20 bp, Artificial
  Synthetic ASO 53
atcttcttttggtttccaca

SEQ ID NO: 54 - DNA, 19 bp, Artificial
  Synthetic ASO 54
ttttgtggtagctcatcac

SEQ ID NO: 55 - DNA, 20 bp, Artificial
  Synthetic ASO 55
ggccacccatctctcctcat

SEQ ID NO: 56 - DNA, 20 bp, Artificial
  Synthetic ASO 56
caccatggatacccatggca

SEQ ID NO: 57 - DNA, 20 bp, Artificial
  Synthetic ASO 57
acgctcttgttgactcttgt

SEQ ID NO: 58 - DNA, 20 bp, Artificial
  Synthetic ASO 58
aattcatttagaaccctgtc

SEQ ID NO: 59 - DNA, 20 bp, Artificial
  Synthetic ASO 59
taggagcttttccatgtttg

SEQ ID NO: 60 - DNA, 20 bp, Artificial
  Synthetic ASO 60
cacatctgtagcaatcagaa

SEQ ID NO: 61 - DNA, 20 bp, Artificial
  Synthetic ASO 61
acatctagccctctggaggc

SEQ ID NO: 62 - DNA, 20 bp, Artificial
  Synthetic ASO 62
tgacaaatttcacatcttcc

SEQ ID NO: 63 - DNA, 20 bp, Artificial
  Synthetic ASO 63
gttagggtagtcataattga

SEQ ID NO: 64 - DNA, 20 bp, Artificial
  Synthetic ASO 64
tgaatataatcctctgagga

SEQ ID NO: 65 - DNA, 20 bp, Artificial
  Synthetic ASO 65
gagcagttcttccaattcga

SEQ ID NO: 66 - DNA, 20 bp, Artificial
  Synthetic ASO 66
tgtgcctgttttggtactgc

SEQ ID NO: 67 - DNA, 20 bp, Artificial
  Synthetic ASO 67
ggtgtaaagaaagtgtatgc

SEQ ID NO: 68 - DNA, 20 bp, Artificial
  Synthetic ASO 68
tcacttgctttatgttatta

SEQ ID NO: 69 - DNA, 20 bp, Artificial
  Synthetic ASO 69
aagcacagagataaggtcgc

SEQ ID NO: 70 - DNA, 20 bp, Artificial
  Synthetic ASO 70
attgcttgattagcttcacg

SEQ ID NO: 71 - DNA, 20 bp, Artificial
  Synthetic ASO 71
actgaagcaacttgggatta

SEQ ID NO: 72 - DNA, 20 bp, Artificial
  Synthetic ASO 72
tgaacctctgtcttcgacca

SEQ ID NO: 73 - DNA, 20 bp, Artificial
  Synthetic ASO 73
cctctacccctggaacgacc

SEQ ID NO: 74 - DNA, 20 bp, Artificial
  Synthetic ASO 74
gacggtcatccttcatgcct

SEQ ID NO: 75 - DNA, 20 bp, Artificial
  Synthetic ASO 75
gcccgcagagtatctgtccc

SEQ ID NO: 76 - DNA, 20 bp, Artificial
  Synthetic ASO 76
gtattaaatccacccctttt

SEQ ID NO: 77 - DNA, 20 bp, Artificial
  Synthetic ASO 77
aattttccctgtctctaaag

SEQ ID NO: 78 - DNA, 20 bp, Artificial
  Synthetic ASO 78
gctagagtaacctctgtcat

SEQ ID NO: 79 - DNA, 20 bp, Artificial
  Synthetic ASO 79
ccaaaatctcttttaagcag

SEQ ID NO: 80 - DNA, 20 bp, Artificial
  Synthetic ASO 80
caccattctgagtttttgcc

SEQ ID NO: 81 - DNA, 19 bp, Artificial
  Synthetic ASO 81
gtaatttgcagcatgtaaa

SEQ ID NO: 82 - DNA, 20 bp, Artificial
  Synthetic ASO 82
cttccaaagctcccattggt

SEQ ID NO: 83 - DNA, 20 bp, Artificial
  Synthetic ASO 83
taccagcagacacaaaatta

SEQ ID NO: 84 - DNA, 20 bp, Artificial
  Synthetic ASO 84
agtcctaaaactggtctgta

SEQ ID NO: 85 - DNA, 20 bp, Artificial
  Synthetic ASO 85
taagtccctgttggattacc

SEQ ID NO: 86 - DNA, 20 bp, Artificial
  Synthetic ASO 86
tgctatcataaccattctgg

SEQ ID NO: 87 - DNA, 20 bp, Artificial
  Synthetic ASO 87
attacttccgtattgctgag

SEQ ID NO: 88 - DNA, 20 bp, Artificial
  Synthetic ASO 88
ccattgtgcatatttggaac

SEQ ID NO: 89 - DNA, 20 bp, Artificial
  Synthetic ASO 89
catatgcctgttggttcata

SEQ ID NO: 90 - DNA, 20 bp, Artificial
  Synthetic ASO 90
agctgcagtagcaggatatg

SEQ ID NO: 91 - DNA, 20 bp, Artificial
  Synthetic ASO 91
ggataaccaatgataggtgc

SEQ ID NO: 92 - DNA, 20 bp, Artificial
  Synthetic ASo 92
gggaatatcctgttggcatt

SEQ ID NO: 93 - DNA, 20 bp, Artificial
  Synthetic ASO 93
tatacttctaaagtcttatt

SEQ ID NO: 94 - DNA, 3769 bp, Homo sapiens
gaccattctccgccgaccaaaacccgtcaaaggattatcagacacgcgggtcggacggtccacatcagccggcagcccgggcgggtcccggggtgcgagcagcgcacttccgtagtgcagcttcggctggtgtcatcggtgtccttcctccgctgccgcccccgcaaggcttcgccgtcatcgaggccatttccagcgacttgtcgcacgcttttctatatacttcgttccccgccaaccgcaaccattgacgccatgtcgggttattcgagtgaccgagaccgcggccgggaccgagggtttggtgcacctcgatttggaggaagtagggcagggcccttatctggaaagaagtttggaaaccctggggagaaattagttaaaaagaagtggaatcttgatgagctgcctaaatttgagaagaatttttatcaagagcaccctgatttggctaggcgcacagcacaagaggtggaaacatacagaagaagcaaggaaattacagttagaggtcacaactgcccgaagccagttctaaatttttatgaagccaatttccctgcaaatgtcatggatgttattgcaagacagaatttcactgaacccactgctattcaagctcagggatggccagttgctctaagtggattggatatggttggagtggcacagactggatctgggaaaacattgtcttatttgcttcctgccattgtccacatcaatcatcagccattcctagagagaggcgatgggcctatttgtttggtgctggcaccaactcgggaactggcccaacaggtgcagcaagtagctgctgaatattgtagagcatgtcgcttgaagtctacttgtatctacggtggtgctcctaagggaccacaaatacgtgatttggagagaggtgtggaaatctgtattgcaacacctggaagactgattgactttttagagtgtggaaaaaccaatctgagaagaacaacctaccttgtccttgatgaagcagatagaatgcttgatatgggctttgaaccccaaataaggaagattgtggatcaaataagacctgataggcaaactctaatgtggagtgcgacttggccaaaagaagtaagacagcttgctgaagatttcctgaaagactatattcatataaacattggtgcacttgaactgagtgcaaaccacaacattcttcagattgtggatgtgtgtcatgacgtagaaaaggatgaaaaacttattcgtctaatggaagagatcatgagtgagaaggagaataaaaccattgtttttgtggaaaccaaaagaagatgtgatgagcttaccagaaaaatgaggagagatgggtggcctgccatgggtatccatggtgacaagagtcaacaagagcgtgactgggttctaaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgaccgtcgggacagatactctgcgggcaaaaggggtggatttaatacctttagagacagggaaaattatgacagaggttactctagcctgcttaaaagagattttggggcaaaaactcagaatggtgtttacagtgctgcaaattacaccaatgggagctttggaagtaattttgtgtctgctggtatacagaccagttttaggactggtaatccaacagggacttaccagaatggttatgatagcactcagcaatacggaagtaatgttccaaatatgcacaatggtatgaaccaacaggcatatgcatatcctgctactgcagctgcacctatgattggttatccaatgccaacaggatattcccaataagactttagaagtatatgtaaatgtctgtttttcataattgctctttatattgtgtgttatctgacaagatagttatttaagaaacatgggaattgcagaaatgactgcagtgcagcagtaattatggtgcactttttcgctatttaagttggatatttctctacattcctgaaacaatttttaggttttttttgtactagaaaatgcaggcagtgttttcacaaaagtaaatgtacagtgatttgaaatacaataaatgaaggcaatgcatggccttccaataaaaaatatttgaagactgaattaagtggaaattgtactttattttatataatgtcatgtaaaactttgcttaagatggtctggttttttttttgtttttgtttggttttttttttccatgaaaacaaatgactgttcctttttatttaatttgggaggcagggggaatcagaaggcccttctttataatgagctattcatattgcaggagtcagaatgaattgatacaggtgaatttttagttacaggctaaattgcataaaagctttgtcagcttccagcatcaggggagtcatttaatagcctttttccttatttgctagtatggttaaatgagaaaatagtaaaatagatacaaagtcatctatatagtgtgagaacgtgggtgactttttcaaagtttataatttaaaaagctccaaataactggctttttcaagagacttatactcatgctcttggctatactgtgaattactgaaatgttgaacaaacctgtgaaagacatacattagccctttaagatggccaggagctaagcttgagtctcctttactgaatttcgttcttagtgcaggttacttgtagattctagtcttcacaggctccctggggctcttaactagtcacactgggagtcatgaatgtctttccaataattcagggaattctagagatcctcaaactgtaaggtctattcatactcaacacaaggaaaaaacctcattaaaattaatgactaatcaggaggcaacgtaaccaaaagcacagtgaatgaaagttttcatggtaggttcaacatgggtttattgctagaaagatccaggggatagctttaggtttaacttcggctcaccaacgtaactttctaatcatttatttcagtaatagctagaagtgggtctgaatgttttcccagagtctgatacgtgtttttttttgccagaagagaggtcttcaggagacttcatttaaattctgattattaaactgaggctttaattgatgttaatgccttatgtcaaatgtaaagttagaatttgctagggctgggatagggagtgatatttctaggacttagacattgaaaactaattcagcctgtagtaacctggatggttttcaatggcatggttagtcaaattcatggttttaaacttagaagcagctttcgggggagagggtaggttggagcatttattacatattttactgtttaatgtcttaaccgtgggccttttaatttgtaaacactgaaatgattgttgggctgtggaaaacatttacctatttaccttggaagttttaaaagacagtccactttttagcatgtgtgttgtgtccagcctgtggtcgtcttaactaataaatgtgatttttctcccca

SEQ ID NO: 95 - DNA, 3662 bp, Homo sapiens
gccattttgatattcacgtcacagtgattggaagagatttgacggtgtagtgtcttcaagcttgctttttgtgtggggattggggagctgtcggggcggctgccatttggtagctgttgagggagttgagagggagcgtattgtgcggatgaaagcgggacgcttcgaggcagacgaaggaacatctgttaggtttggtgcacctcgatttggaggaagtagggcagggcccttatctggaaagaagtttggaaaccctggggagaaattagttaaaaagaagtggaatcttgatgagctgcctaaatttgagaagaatttttatcaagagcaccctgatttggctaggcgcacagcacaagaggtggaaacatacagaagaagcaaggaaattacagttagaggtcacaactgcccgaagccagttctaaatttttatgaagccaatttccctgcaaatgtcatggatgttattgcaagacagaatttcactgaacccactgctattcaagctcagggatggccagttgctctaagtggattggatatggttggagtggcacagactggatctgggaaaacattgtcttatttgcttcctgccattgtccacatcaatcatcagccattcctagagagaggcgatgggcctatttgtttggtgctggcaccaactcgggaactggcccaacaggtgcagcaagtagctgctgaatattgtagagcatgtcgcttgaagtctacttgtatctacggtggtgctcctaagggaccacaaatacgtgatttggagagaggtgtggaaatctgtattgcaacacctggaagactgattgactttttagagtgtggaaaaaccaatctgagaagaacaacctaccttgtccttgatgaagcagatagaatgcttgatatgggctttgaaccccaaataaggaagattgtggatcaaataagacctgataggcaaactctaatgtggagtgcgacttggccaaaagaagtaagacagcttgctgaagatttcctgaaagactatattcatataaacattggtgcacttgaactgagtgcaaaccacaacattcttcagattgtggatgtgtgtcatgacgtagaaaaggatgaaaaacttattcgtctaatggaagagatcatgagtgagaaggagaataaaaccattgtttttgtggaaaccaaaagaagatgtgatgagcttaccagaaaaatgaggagagatgggtggcctgccatgggtatccatggtgacaagagtcaacaagagcgtgactgggttctaaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgaccgtcgggacagatactctgcgggcaaaaggggtggatttaatacctttagagacagggaaaattatgacagaggttactctagcctgcttaaaagagattttggggcaaaaactcagaatggtgtttacagtgctgcaaattacaccaatgggagctttggaagtaattttgtgtctgctggtatacagaccagttttaggactggtaatccaacagggacttaccagaatggttatgatagcactcagcaatacggaagtaatgttccaaatatgcacaatggtatgaaccaacaggcatatgcatatcctgctactgcagctgcacctatgattggttatccaatgccaacaggatattcccaataagactttagaagtatatgtaaatgtctgtttttcataattgctctttatattgtgtgttatctgacaagatagttatttaagaaacatgggaattgcagaaatgactgcagtgcagcagtaattatggtgcactttttcgctatttaagttggatatttctctacattcctgaaacaatttttaggttttttttgtactagaaaatgcaggcagtgttttcacaaaagtaaatgtacagtgatttgaaatacaataaatgaaggcaatgcatggccttccaataaaaaatatttgaagactgaattaagtggaaattgtactttattttatataatgtcatgtaaaactttgcttaagatggtctggttttttttttgtttttgtttggttttttttttccatgaaaacaaatgactgttcctttttatttaatttgggaggcagggggaatcagaaggcccttctttataatgagctattcatattgcaggagtcagaatgaattgatacaggtgaatttttagttacaggctaaattgcataaaagctttgtcagcttccagcatcaggggagtcatttaatagcctttttccttatttgctagtatggttaaatgagaaaatagtaaaatagatacaaagtcatctatatagtgtgagaacgtgggtgactttttcaaagtttataatttaaaaagctccaaataactggctttttcaagagacttatactcatgctcttggctatactgtgaattactgaaatgttgaacaaacctgtgaaagacatacattagccctttaagatggccaggagctaagcttgagtctcctttactgaatttcgttcttagtgcaggttacttgtagattctagtcttcacaggctccctggggctcttaactagtcacactgggagtcatgaatgtctttccaataattcagggaattctagagatcctcaaactgtaaggtctattcatactcaacacaaggaaaaaacctcattaaaattaatgactaatcaggaggcaacgtaaccaaaagcacagtgaatgaaagttttcatggtaggttcaacatgggtttattgctagaaagatccaggggatagctttaggtttaacttcggctcaccaacgtaactttctaatcatttatttcagtaatagctagaagtgggtctgaatgttttcccagagtctgatacgtgtttttttttgccagaagagaggtcttcaggagacttcatttaaattctgattattaaactgaggctttaattgatgttaatgccttatgtcaaatgtaaagttagaatttgctagggctgggatagggagtgatatttctaggacttagacattgaaaactaattcagcctgtagtaacctggatggttttcaatggcatggttagtcaaattcatggttttaaacttagaagcagctttcgggggagagggtaggttggagcatttattacatattttactgtttaatgtcttaaccgtgggccttttaatttgtaaacactgaaatgattgttgggctgtggaaaacatttacctatttaccttggaagttttaaaagacagtccactttttagcatgtgtgttgtgtccagcctgtggtcgtcttaactaataaatgtgatttttctcccca